(19) United States
(12) Patent Application Publication
(10) Pub. No.: US 2009/0047699 A1
(43) Pub. Date: Feb. 19, 2009

(54) FLUOROGENIC ENZYME ACTIVITY ASSAY METHODS AND COMPOSITIONS USING FRAGMENTABLE LINKERS

(75) Inventor: Ronald J. Graham, San Ramon, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLIED BIOSYSTEMS INC., Foster City, CA (US)

(21) Appl. No.: 12/188,165

(22) Filed: Aug. 7, 2008

Related U.S. Application Data
(63) Continuation of application No. 11/147,827, filed on Jun. 7, 2005, now abandoned.
(60) Provisional application No. 60/577,995, filed on Jun. 7, 2004.

(51) Int. Cl. C12Q 1/34; C07D 311/82

(52) U.S. Cl. 435/23; 549/223

(57) ABSTRACT

Substrate compound-containing micelles and various compositions, kits and methods for their preparation and use are provided.

### 1. CROSS-REFERENCES TO RELATED APPLICATIONS

This application is a continuation of U.S. patent application Ser. No. 11/147,827, filed Jun. 7, 2005 which claims benefit under 35 U.S.C. § 119(e) to application No. 60/577,995, entitled “Fluorogenic Enzyme Activity Assay Methods and Compositions Using Fragmentable Linkers”, filed Jun. 7, 2004; both of which are incorporated herein by reference in its entirety.

### 2. FIELD

The present disclosure relates to fluorescent compositions and methods for detecting or characterizing target agents.

### 3. INTRODUCTION

Assays using reporter molecules are important tools for studying and detecting molecules that mediate numerous biological and industrial processes. For example, enzymes perform a multitude of biological tasks, such as synthesis and replication of nucleic acids, modification, and degradation of polypeptides, synthesis of metabolites, and many other functions. Enzymes are also used in industry for many purposes, such as proteases used in laundry detergents, metabolic enzymes to make specialty chemicals such as amino acids and vitamins, and chirally specific enzymes to prepare enantiomerically pure drugs. In medical testing, enzymes are important indicators of the health or disease of human patients. Reporter molecules also can be used to detect conditions associated with disease states, such as hypoxic regions characteristic of solid tumors. Although numerous approaches have been developed for assaying enzymes, as well as other target agents, there is still a great need to find new assay designs that can be used to inexpensively and conveniently detect and characterize a wide variety of enzymes.

### 4. SUMMARY

Provided herein are substrate compounds useful for, among other things, detecting the presence and/or quantity of a molecule of interest. The substrate compound comprises at least one hydrophobic moiety capable of integrating the substrate compound into a micelle, a fluorescent moiety, a trigger moiety and a linker moiety linking the hydrophobic moiety, the fluorescent moiety and the trigger moiety together. The substrate compound can be incorporated into a micelle and subjected to conditions effective to allow activation of the trigger moiety by a trigger agent. Activation of the trigger moiety initiates a spontaneous rearrangement that results in the fragmentation of the substrate compound to release either the fluorescent moiety or the hydrophobic moiety, thereby increasing the fluorescent signal produced by the fluorescent moiety.

The micelles comprise a detection system that permits the micelles to be selectively “turned on” by treatment with specified trigger agents. The micelles can exist in a variety of different forms, ranging from non-lamellar “detergent-like” micelles which do not enclose or encapsulate solvent, to lamellar vesicle-like micelles which do enclose or encapsulate solvent (e.g., aqueous solvent), such as, for example, liposomes. The lamellar vesicle-like micelles may be unilamellar or multilamellar, and may vary in size, ranging from small to large. In some embodiments, such micelles comprise small unilamellar vesicles or liposomes (“SUVs”), small multilamellar vesicles or liposomes (SMVs”), large unilamellar vesicles or liposomes (“LUVs”) and/or large multilamellar vesicles or liposomes (“LMVs”). A collection of micelles may all be of the same type or, alternatively, may comprise mixtures of two or more of the various different micellar forms. Vesicle-like micelles may be unfilled, or all or a subset of them may encapsulate or enclose a substrate compound, a quencher molecule or a mixture thereof.

The substrate compound-containing micelles generally comprise one or more substrate compounds capable of generating or providing a detectable fluorescent signal under specified conditions. For example, in some embodiments, the micelles can comprise two or more substrate compounds. In embodiments comprising two or more substrate compounds, the substrate compounds can be the same, some can be the same and others different, or they all can differ from each other. The substrate compound comprises a trigger moiety, at least one hydrophobic moiety, a fluorescent moiety, and a linker moiety capable of undergoing fragmentation.

The trigger moiety can comprise any substrate that when acted on by a trigger agent is capable of generating an intermediate compound that spontaneously rearranges resulting in fragmentation of the substrate compound. In some embodiments, fragmentation results in the release of the fluorescent moiety from the substrate compound. In other embodiments, fragmentation results in the release of the hydrophobic moiety from the substrate compound. Regardless of whether the fluorescent moiety or the hydrophobic moiety is released, the fluorescent signal produced by the fluorescent moiety is increased, indicating the presence of the molecule of interest in the sample.

The chemical structure of the trigger moiety will depend, in part, upon the particular trigger agent. In some embodiments, the trigger moiety comprises a cleavage site that is recognized and cleaved by a cleaving enzyme. For example, the cleaving enzyme can be a lipase, an esterase, a phosphatase, a glycosidase, a carboxypeptidase or a catalytic antibody. In some embodiments, the trigger moiety comprises an oligonucleotide or oligonucleotide analog having a sequence that is recognized and cleaved by a nuclease, such as a ribonuclease or a deoxyribonuclease. In some embodiments, the trigger moiety comprises a peptide or peptide analog that is recognized and cleaved by a protease.

In some embodiments, the trigger moiety comprises a cleavage site comprising a phosphate moiety that is capable of being hydrolyzed by a phosphatase. The trigger moiety may also comprise additional residues that facilitate specificity, affinity and/or rate of hydrolysis of the particular phosphatase. The trigger moiety may be designed to be recognized by a particular phosphatase or group of phosphatases.

In some embodiments, the trigger moiety comprises a cleavage site comprising one or more carbohydrates that are capable of being hydrolyzed by a glycosidase, such as β-galactosidase or β-glucoronidase. The trigger moiety may also comprise additional residues that facilitate specificity, affinity and/or rate of hydrolysis of the particular glycosidase. The trigger moiety may be designed to be recognized and hydrolyzed by a particular glycosidase or group of glycosidases.

In some embodiments, the trigger moiety comprises a cleavage site comprising esters of glycerol and fatty acids that are capable of being hydrolyzed by a lipase, such as triacylglycerol lipase. The trigger moiety may also comprise additional residues that facilitate specificity, affinity and/or rate of hydrolysis of the particular lipase. The trigger moiety may be designed to be recognized and hydrolyzed by a particular lipase or group of lipases.

In some embodiments, the trigger moiety comprises a cleavage site comprising an ester moiety that is capable of being hydrolyzed by an esterase. The trigger moiety may also comprise additional residues that facilitate specificity, affinity and/or rate of hydrolysis of the particular esterase. The trigger moiety may be designed to be recognized and hydrolyzed by a particular esterase or group of esterases.

In some embodiments, the trigger moiety comprises a cleavage site comprising a peptide bond, a peptide analog, or a peptide sequence that is capable of being hydrolyzed by a protease, such as carboxypeptidase A, carboxypeptidase G2, protease plasmin, trypsin, proteases such as serine, cysteine, aspartyl and metalloproteases. For example, in some embodiments the trigger moiety comprises a peptide sequence or peptide analog that is recognized and cleaved by a protease. In other embodiments, the trigger moiety comprises cleavage site comprising an amidic, urethanic, or ureidic bond connecting the linker moiety to an amino acid. The trigger moiety may also comprise additional residues that facilitate specificity, affinity and/or rate of hydrolysis of the particular protease. The trigger moiety may be designed to be recognized and hydrolyzed by a particular protease or group of proteases.

In some embodiments, the trigger moiety comprises a cleavage site comprising a transition state analogue to which a catalytic antibody has been raised. For example, N-methylcarbamate can be attached to a carrier protein and used as a transition state analogue to which catalytic antibodies can be raised. Hydrolysis of N-methylcarbamate by the catalytic antibody results in fragmentation of the substrate compound and release of the hydrophobic moiety or the fluorescent moiety. The trigger moiety may also comprise additional residues that facilitate specificity, affinity and/or rate of hydrolysis of the particular catalytic antibody.

In addition to having a cleavage site for a cleaving enzyme, the trigger moiety may include additional linkages that facilitate the attachment of the cleavage site to the substrate compound. In these embodiments, the additional linkages are capable of undergoing spontaneous rearrangement such that fragmentation of the substrate compound results.

In other embodiments, reduction of an aromatic nitro or azide compound can be used as a bioreductive trigger agent to generate a π electron-donor species, e.g. —NH—, that is capable of initiating a spontaneous rearrangement reaction, resulting in fragmentation of the substrate compound.

In other embodiments, the trigger moiety is also the linker moiety. In these embodiments, cleavage of the trigger moiety results directly in the release of the hydrophobic moiety or the fluorescent moiety. For example, if the linker moiety is a substrate for β-lactamase, cleavage of the linker moiety by β-lactamase initiates a fragmentation reaction that results in the release of either the hydrophobic moiety or the fluorescent moiety.

The hydrophobic moiety(ies) are selected such that, taken together, they are capable of integrating the substrate compound into a micelle. In some embodiments, each hydrophobic moiety comprises a saturated or unsaturated hydrocarbon comprising from 6 to 30 carbon atoms. When a substrate molecule comprises more than one hydrophobic moiety, the hydrophobic moieties may be the same, some of them may be the same and others different, or they may all differ from one another. In some embodiments, the substrate molecule comprises two hydrophobic moieties, each of which comprises a hydrocarbon chain corresponding in structure to a hydrocarbon chain or “tail” of a naturally occurring lipid or phospholipid.

In some embodiments, the release of the hydrophobic moiety(ies) facilitates an increase in the fluorescence of the fluorescent moiety following fragmentation of the substrate compound such that the intensity of the fluorescence following fragmentation is greater than would be obtained with the same substrate compound lacking the hydrophobic moiety(ies).

The fluorescent moiety may be any fluorescent entity that is operative in accordance with the various compositions and methods described herein. In some embodiments, the fluorescent moiety comprises at least one fluorescein dye. In some embodiments, the fluorescent moiety comprises at least one rhodamine dye. In some embodiments, the fluorescent moiety comprises two or more fluorescent dyes that can act cooperatively with one another, such as by, for example, fluorescence resonance energy transfer (“FRET”).

In some embodiments, the fluorescence of the fluorescent moiety is quenched as result of integration of the substrate compound in the micelle. This quenching may be accomplished by a variety of different mechanisms. In some embodiments, the substrate compound comprises a fluorescent moiety that is capable of “self-quenching” when in close proximity to another fluorescent moiety of the same type. In such embodiments, the micelle may comprise substrate compounds in an amount or concentration high enough to bring the fluorescent moieties of different substrate compounds in sufficiently close proximity to one another such that the fluorescence of their fluorescent moieties is quenched.

In some embodiments, quenching can be achieved with the aid of a quenching moiety. The quenching moiety can be any moiety capable of quenching the fluorescence of the fluorescent moiety of a substrate compound when it is in close proximity thereto, such as, for example, by orbital overlap (formation of a ground state dark complex), collisional quenching, FRET, photoinduced electron transfer (PET) or another mechanism or combination of mechanisms. The quenching moiety can itself be fluorescent, or it can be non-fluorescent. In some embodiments, the quenching moiety comprises a fluorescent dye that has an absorbance spectrum that sufficiently overlaps the emissions spectrum of the fluorescent moiety of the substrate compound such that it quenches the fluorescence of the fluorescent moiety when in close proximity thereto. In such embodiments, selecting a quenching moiety that fluoresces at a wavelength resolvable from that of the fluorescent moiety can provide an internal signal standard to which the fluorescence signal can be referenced and also permits the micelles to be “tracked” by the fluorescence of the quenching moiety.

The quenching moiety can be included in a distinct quenching molecule that has properties that permit it to integrate into the micelle to quench the fluorescence of the fluorescent moieties of the substrate compounds. In some embodiments, a quenching molecule comprises at least one hydrophobic moiety, such as one of the hydrophobic moiety(ies) described above, and a quenching moiety. The quenching molecule can optionally comprise a linker moiety, as will be described in more detail below. When the quenching molecule comprises an optional linker moiety, fragmentation of the linker moiety following activation of the trigger moiety by the trigger agent leads to unquenching of the fluorescent moieties of the substrate compounds.

The hydrophobic moiety, fluorescent moiety and trigger moiety of the substrate compound can be connected to the linker moiety in any way that permits them to perform their respective functions. The connectivities may depend, in part, upon the mechanism used to fragment the substrate compound. In some embodiments, the trigger moiety is linked to the linker moiety directly via a strong π electron-donor moiety, while in other embodiments the trigger moiety is linked to the π electron-donor moiety indirectly via additional linkages. In some embodiments, the fluorescent or hydrophobic moiety is linked to the linker moiety via a linkage that comprises a moiety that is capable of “leaving” upon fragmentation of the substrate compound. In other embodiments, the fluorescent or hydrophobic moiety is linked to the linker via a stable linkage that does not dissociate from the backbone of the substrate compound following the fragmentation reaction.

Regardless of the mechanism by which the quenching effect is achieved, fragmentation of the substrate compound leads to unquenching of the fluorescence signal, thereby producing a detectable change in fluorescence. The mechanism by which the fragmentation leads to unquenching is not critical, and can be selected by the user, depending, in part, on the particular application. For example, fragmentation reactions can be based on electronic cascade self-elimination reactions, and can include electronic cascade fragmentable linker moieties that self-eliminate through linear or cyclic 1,4-, 1,6- or 1,8-elimination reaction. In other embodiments, fragmentation of the substrate compound may be based on a ring closure mechanism, such as an intramolecular cyclization reaction or a trimethyl lock lactonization reaction. The fragmentation systems described herein are designed to release either a fluorescent moiety or a hydrophobic moiety as a result of fragmentation of the substrate compound.

The chemical structure of the linker moiety can be selected by the user, depending, in part, upon the particular fragmentation reaction. Any molecule having two, three, four, or more attachment sites suitable for attaching other molecules and moieties thereto, or that can be appropriately activated to attach other molecules and moieties thereto, could be used. For example, the “backbone” of the linker can have two sites of attachment, such that the hydrophobic moiety can be attached at one end and the fluorescent moiety attached to the other end. An exemplary example of a “linear” linker is β-lactam. In other embodiments, the linker moiety can comprise a five or six-membered aromatic ring, such as a phenyl ring or a benzyl ring, a heterocyclic ring with nitrogen, oxygen, sulfur or phosphorus, or an aryl group or heterocyclic ring comprising multiple sites, e.g., two, three, four, five or more sites, for the attachment of the trigger moiety, the hydrophobic moiety, the fluorescent moiety and one more substituents. Suitable substituents include, but are not limited to, halogens such as chlorine and fluorine, amino groups, hydroxy groups, carboxylic acids, nitro groups, and alkyl groups such as methyl, etc.

In embodiments in which fragmentation occurs via a 1,4-, 1,6-, or 1,8-elimination reaction, the “backbone” of the linker moiety can comprise a benzyl group bearing sites for the attachment of the trigger moiety, the fluorescent moiety, the hydrophobic moiety, and one or more substituents. Typically, the attachment site for the trigger moiety comprises a π electron-donor moiety with optional linkages. Linkages can also be used to attach the hydrophobic moiety, the fluorescent moiety, and optional substituents to the backbone of the linker moiety.

The linkages can be any moiety to which the trigger moiety, hydrophobic moiety(ies) and fluorescent moiety can be attached and which permit the trigger moiety, hydrophobic moiety(ies) and fluorescent moiety to perform their respective functions. The composition of the linkage will vary depending on the nature of the moiety. For example, linkages can be selected to control the rate of the cleavage reaction by the trigger agent. Other types of linkages useful in the compositions and methods described herein include stable linkages, and linkages comprising leaving groups. For example, the fluorescent moiety can be attached through a linkage comprising a leaving group, while the hydrophobic moiety can be attached through a stable linkage, e.g., a linkage that does not comprise a leaving group. Alternatively, the fluorescent moiety can be attached through a stable linkage, e.g., a linkage that does not comprise a leaving group, while the hydrophobic moiety can be attached through a linkage comprising a leaving group. In other embodiments, the linkages used to attach the fluorescent moiety and the hydrophobic moiety can be the same. Examples of suitable linkages for use in the compositions and methods described herein are discussed below.

Fragmentation of the substrate compound also can occur via a ring closure mechanism. In some embodiments, the central core of the linker moiety can comprise a phenyl compound bearing a strong π electron-donor moiety attached to the carbon atom at position C1 of the phenyl ring. A trigger moiety can be directly or indirectly attached to the strong π electron-donor moiety. The fluorescent moiety can be attached to the carbon atom at position C2 of the phenyl ring via a linkage comprising a derivative of propionic acid, such as β,β-dimethylpropionic acid amide, and a leaving group, while the hydrophobic moiety can be attached to the carbon atom at position C5 via a stable linkage that does not dissociate from the backbone of the phenyl linker upon fragmentation. Conversely, the hydrophobic moiety can be attached to the carbon atom at position C2 of the phenyl ring via a linkage comprising a derivative of propionic acid, such as β,β-dimethylpropionic acid amide, and a leaving group, while the fluorescent moiety can be attached to the carbon atom at position C5 via a stable linkage that does not dissociate from the backbone of the phenyl linker upon fragmentation. Cleavage of the trigger moiety by a trigger agent regenerates the hydroxy or amino group at the carbon atom at the C1 position of the phenyl ring. The hydroxy or amino group then initiates the ring closure mechanism, which leads to the release of the hydrophobic moiety or the fluorescent moiety, depending on which moiety is attached to the leaving group.

Also provided are methods that utilize the substrate compound-containing micelles such as discussed above. In some embodiments, a method is provided for detecting the presence and/or quantity of a molecule of interest in a sample that comprises the steps of:

(a) contacting the sample with a micelle comprising a substrate compound comprising a hydrophobic moiety, a fluorescent moiety, a trigger moiety and a linker moiety under conditions in which the trigger moiety is triggered, either directly or indirectly, by the target agent if present in the sample;

(b) detecting a fluorescence signal, where an increase in the fluorescence signal indicates the presence and/or quantity of the target agent in the sample.

In some embodiments of such methods, the micelle further comprises a quenching molecule comprising a quenching moiety capable of quenching the fluorescence of the fluorescent moiety of the substrate compound when in close proximity thereto, and at least one moiety capable of integrating the quenching molecule into the micelle. For example, in some embodiments, the quenching molecule can comprise a hydrophobic moiety capable of integrating the quenching molecule into the micelle. In other embodiments, hydrophobicity can be conferred by attaching a pyrene or lipid soluble dye to the quenching molecule.

As another example, the micelles and methods can be used to screen for and/or identify a molecule of interest. For example, a plurality of micelles may be prepared, each of which comprises a different substrate compound and contacted with one or more samples to identify a molecule of interest. Such screening assays may be carried out in a “single-plex” mode in which each micelle of the plurality is contacted individually with the molecule of interest, or in a “multiplex” mode in which all or a subset of the micelles are contacted simultaneously with the molecule of interest. In some embodiments of such multiplexed assays, each micelle can comprise a fluorescent moiety having a fluorescence spectrum or signal that is resolvable from the fluorescence spectra or signals of the fluorescent moieties of the substrate compounds of the other micelles such that the identities of putative target agents can be correlated with a specified fluorescence signal or “color”.

In another aspect, the present disclosure provides substrate compounds, quenching molecules, substrate compound-containing micelles and kits containing the substrate compounds, quenching molecules, substrate compound-containing micelles as discussed further herein.

These and other features of the compositions and methods described herein will become apparent from the detailed description below.

### 5. BRIEF DESCRIPTION OF THE DRAWINGS

Aspects of the various embodiments described herein can be more fully understood with respect to the following drawings.

FIGS. 1A-1D illustrate the release of a dye moiety or a hydrophobic moiety following fragmentation of the substrate compound;

FIG. 2A illustrates an exemplary embodiment of a substrate compound in which the trigger moiety also serves as the linker moiety;

FIG. 2B illustrates an exemplary embodiment of a substrate compound comprising an aromatic linker moiety that fragments via 1,6-elimination reaction and the resulting fragmentation products;

FIGS. 3A-3D illustrate exemplary embodiments of substrate compounds comprising linker moieties that fragment via a trimethyl lock lactonization reaction and the resulting fragmentation products;

FIGS. 4A-4B illustrate exemplary embodiments of substrate compounds comprising linker moieties that fragment via a ring closure mechanism and the resulting fragmentation products;

FIGS. 5A-5B illustrate an exemplary method of synthesizing a substrate compound that comprises a linker moiety that fragments via a 1,6-elimination reaction;

FIG. 6 illustrates an exemplary method of synthesizing a substrate compound that comprises a linker moiety that fragments via a 1,6-elimination reaction;

FIG. 7 illustrates another exemplary method of synthesizing a substrate compound that comprises a linker moiety that fragments via a 1,6-elimination reaction;

FIGS. 8A-8B illustrates another exemplary method of synthesizing a substrate compound that comprises a linker moiety that fragments via a 1,4- and a 1,6-elimination reaction;

FIGS. 9A-9B illustrates an exemplary method of synthesizing a substrate compound that comprises a linker moiety that fragments via a bis 1,4-elimination reaction;

FIGS. 10A-10E illustrate other exemplary methods of synthesizing a substrate compound that comprises a linker moiety that fragments via a 1,6-elimination reaction; and,

FIGS. 11A-11B illustrate an exemplary method of synthesizing a substrate compound that comprises a linker moiety that fragments via a ring closure mechanism.

### 6. DETAILED DESCRIPTION

It is to be understood that both the foregoing general description and the following detailed description are exemplary and explanatory only and are not restrictive of the embodiments described herein. In this application, the use of the singular includes the plural unless specifically stated otherwise. Also, the use of “or” means “and/or” unless stated otherwise. Similarly, “comprise,” “comprises,” “comprising,” “include,” “includes” and “including” are not intended to be limiting.

### 6.1 Definitions

As used herein, the following terms and phrases are intended to have the following meanings:

“Detect” and “detection” have their standard meaning, and are intended to encompass detection, measurement, and/or characterization of a selected molecule or molecular activity. For example, enzyme activity may be “detected” in the course of detecting or screening for an enzyme capable of recognizing and cleaving a defined/specified/known cleavage site.

“Fatty Acid” has its standard meaning and is intended to refer to a long-chain hydrocarbon carboxylic acid in which the hydrocarbon chain is saturated, mono-unsaturated or polyunsaturated. The hydrocarbon chain may be linear, branched or cyclic, or may comprise a combination of these features, and may be unsubstituted or substituted. Fatty acids typically have the structural formula RC(O)OH, where R is a substituted or unsubstituted, saturated, mono-unsaturated or polyunsaturated hydrocarbon comprising from 6 to 30 carbon atoms which has a structure that is linear, branched, cyclic or a combination thereof.

“Phospholipid” has its standard meaning and is intended to include compounds which comprise two fatty acid moieties, a backbone moiety, a phosphate moiety, and an organic moiety. Specific examples of phospholipids include glycerophospholipids and sphingolipids. Specifically included within the definition of “phospholipid” are glycerophospholipids having the following structure:

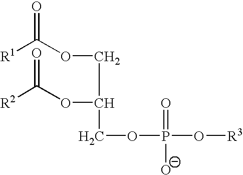

wherein:

R¹ is a saturated, mono-unsaturated or polyunsaturated hydrocarbon having from 6 to 30 carbon atoms;

R² is a saturated, mono-unsaturated or polyunsaturated hydrocarbon having from 6 to 30 carbon atoms; and

R³ is —CH₂CH₂—N⁺(CH₃)₃ (cholinyl), —CH₂CH₂NH₂ (ethanolamin-2-yl), inositolyl, —CH₂CH(NH₃⁺)C(O)OH (serinyl) or —CH₂CH(NH₂)—CH(OH)—CH═CH—(CH₂)₁₂CH₃ (sphingosinyl).

“Micelle” has its standard meaning and is intended to refer to an aggregate formed by amphipathic molecules in water or an aqueous environment such that their polar ends or portions are in contact with the water or aqueous environment and their nonpolar ends or portions are in the interior of the aggregate. A micelle can take any shape or form, including but not limited to, a non-lamellar “detergent-like” aggregate that does not enclose a portion of the water or aqueous environment, or a unilamellar or multilamellar “vesicle-like” aggregate that encloses a portion of the water or aqueous environment, such as, for example, a liposome.

“Quench” has its standard meaning and is intended to refer to a reduction in the fluorescence intensity of a fluorescent group or moiety as measured at a specified wavelength, regardless of the mechanism by which the reduction is achieved. As specific examples, the quenching may be due to molecular collision, energy transfer such as FRET, photoinduced electron transfer such as PET, a change in the fluorescence spectrum (color) of the fluorescent group or moiety or any other mechanism (or combination of mechanisms). The amount of the reduction is not critical and may vary over a broad range. The only requirement is that the reduction be detectable by the detection system being used. Thus, a fluorescence signal is “quenched” if its intensity at a specified wavelength is reduced by any measurable amount. A fluorescence signal is “substantially quenched” if its intensity at a specified wavelength is reduced by at least 50%, for example by 50%, 60%, 70%, 75%, 80%, 85%, 90%, 95%, 96%, 97%, 98%, 99% or even 100%.

Polypeptide sequences are provided with an orientation (left to right) of the N terminus to C terminus, with amino acid residues represented by the standard 3-letter or 1-letter codes (e.g., Stryer, L., Biochemistry, 2^{nd} Ed., W.H. Freeman and Co., San Francisco, Calif., page 16 (1981)).

### 6.2 Exemplary Embodiments

Provided herein are compositions, methods and kits that utilize substrate compound-containing micelles. The substrate compound-containing micelles comprise as one component a substrate compound comprising a fluorescent moiety, at least one hydrophobic moiety, a trigger moiety and a linker moiety that is capable of fragmenting following an electronic cascade self-elimination reaction. In some embodiments, the trigger moiety comprises a substrate that can be cleaved by a specified trigger agent. The fluorescent moiety, the hydrophobic moiety(ies), and the trigger moiety are connected to the linker moiety in any way that permits them to perform their respective functions. The fluorescence signal of the fluorescent moiety is quenched when the substrate compound is integrated into the micelle. Activation of the trigger moiety by the specified trigger agent eliminates the quenching effect, thereby producing a detectable increase in fluorescence. Suitable activation events include, but are not limited to, enzymatic cleavage, or bioreduction of the trigger moiety.

In some embodiments, activation of the trigger moiety results in the release of the fluorescent moiety from the micelle, thereby reducing or eliminating the quenching effect caused by the interactions between the fluorescent moiety and the micelle. The release may be caused by a 1,4-, a 1,6-, or a 1,8-elimination reaction that fragments the substrate compound such that the fluorescent moiety is released from the “backbone” of the linker moiety. The release may also be caused by a ring closure mechanism that fragments the substrate compound such that the fluorescent moiety is released from the “backbone” of the linker. Regardless of the mechanism used to release the fluorescent moiety, the fluorescent signal produced by the fluorescent moiety is increased, indicating the presence of the specified trigger agent in the sample.

In other embodiments, activation of the trigger moiety results in the release of the hydrophobic moiety from the backbone of the linker moiety, thereby releasing the fragment of the substrate compound comprising the fluorescent moiety from the micelle. Release of the fluorescent moiety from the micelle, reduces or eliminates the quenching effect caused by the interactions between the fluorescent moiety and the micelle. Release of the hydrophobic moiety may be caused by a 1,4-, a 1,6-, or a 1,8-elimination reaction that fragments the substrate compound such that the hydrophobic moiety is released from the “backbone” of the linker moiety. The release may also be caused by a ring closure mechanism that fragments the substrate compound such that the hydrophobic moiety is released from the “backbone” of the linker moiety.

In other embodiments, the substrate compound-containing micelle comprises a substrate compound as one component and a quenching molecule as another component. The substrate compound comprises at least one hydrophobic moiety capable of integrating the substrate compound into the micelle, a fluorescent moiety, a trigger moiety and a linker moiety. The quenching molecule comprises at least one hydrophobic moiety capable of integrating the quenching molecule into the micelle and a quenching moiety capable of quenching the fluorescence of the fluorescent moiety of the substrate compound when in close proximity thereto. The hydrophobic moiety can comprise a substituted or unsubstituted hydrocarbon, a pyrene, or a lipid soluble dye. The quenching molecules may optionally comprise a trigger moiety that can be activated by a specified trigger agent and a linker moiety. When both the substrate compound and quenching molecules comprise a trigger moiety, they can be activated by the same trigger agent, or by different trigger agents. The various moieties of the substrate compound and quenching molecules are connected in any way that permits them to perform their respective functions. Fragmentation of the linker reduces or eliminates the quenching effect, by relieving their close proximity, thereby producing a detectable increase in fluorescence. Suitable types of fragmentation events include those described above.

The substrate compound-containing micelles described herein can be used as selectively activatable dyes to detect target agents. The micelles may also be used to screen and/or identify agents that are associated with a particular organism or disease state. The organism may be eukaryotic or prokaryotic pathogenic or non-pathogenic. The disease state can be any disease of interest. For instance, proteases associated with cancer could be screened for and identified using the compositions and methods described herein.

### 6.3 The Substrate Compound

The substrate compounds typically comprise one or more hydrophobic moieties capable of anchoring or integrating the substrate compound into the micelle. The exact numbers, lengths, sizes and/or composition of the hydrophobic moieties can be selectively varied. In one embodiment, the hydrophobic moiety comprises a substituted or unsubstituted hydrocarbon of sufficient hydrophobic character (e.g., length and/or size) to cause the substrate compound to become integrated or incorporated into a micelle when the substrate compound is placed in an aqueous environment at a concentration above a micelle-forming threshold, such as at or above its critical micelle concentration (CMC). The number of hydrophobic moieties comprising a micelle is not critical and can vary, as long as the number of hydrophobic moieties is sufficient to quench the fluorescence of the fluorescent moiety(ies) in the absence of a trigger agent. For example, in some embodiments a dimer comprising two hydrophobic moieties is sufficient to quench the fluorescence of the fluorescent moieties in the absence of a trigger agent. In other embodiments, more than two hydrophobic moieties may be necessary to detect a measurable difference in fluorescence following release of the fluorescent moiety from the substrate compound. For any substrate compound, the number of hydrophobic moieties required can be determined empirically by measuring fluorescence as a function of substrate concentration before and after the addition of a trigger agent.

In another embodiment, the hydrophobic moiety comprises a substituted or unsubstituted hydrocarbon comprising from 6 to 30 carbon atoms, or from 6 to 25 carbon atoms, or from 6 to 20 carbon atoms, or from 6 to 15 carbon atoms, or from 8 to 30 carbon atoms, or from 8 to 25 carbon atoms, or from 8 to 20 carbon atoms, or from 8 to 15 carbon atoms, or from 12 to 30 carbon atoms, or from 12 to 25 carbon atoms, or from 12 to 20 carbon atoms. The hydrocarbon may be linear, branched, cyclic, or any combination thereof. Exemplary hydrocarbon groups comprise C6, C7, C8, C9, C10, C11, C12, C13, C14, C15, C16, C17, C18, C19, C20, C22, C24, and C26 alkyl chains.

In some embodiments, the hydrophobic moiety is fully saturated. In some embodiments, the hydrophobic moiety can comprise one or more carbon-carbon double bonds which may be, independently of one another, in the cis or trans configuration, and/or one or more carbon-carbon triple bonds. In some cases, the hydrophobic moiety may have one or more cycloalkyl groups, or one or more aryl rings or arylalkyl groups, such as one or two phenyl rings.

As will be described in more detail below, in some embodiments the substrate compound is an analog or a derivative of a glycerophospholipid. In such embodiments, the substrate compound typically comprises two hydrophobic moieties linked to the C1 and C2 carbons of a glycerolyl group via ester linkages (or other linkages). The two hydrophobic moieties may be the same or they may differ from another. In a specific embodiment, each hydrophobic moiety corresponds to the hydrocarbon chain or “tail” of a naturally occurring fatty acid. In another specific embodiment, the hydrophobic moieties correspond to the hydrocarbon chains or tails of a naturally occurring phospholipid. Non-limiting examples of hydrocarbon chains or tails of commonly occurring fatty acids are provided in Table 1, below:

TABLE 1

| Length:Number of Unsaturations | Common Name |
|---|---|
| 14:0 | myristic acid |
| 16:0 | palmitic acid |
| 18:0 | Stearic acid |
| 18:1 cisΔ⁹ | oleic acid |
| 18:2 cisΔ^{9, 12} | Linoleic acid |
| 18:3 cisΔ^{9, 12, 15} | linonenic acid |
| 20:4 cisΔ^{5, 8, 11, 14} | arachidonic acid |
| 20:5 cisΔ^{5, 8, 11, 14, 17} | eicosapentaenoic acid (an omega-3 |
| | fatty acid) |

The substrate compound further comprises a fluorescent moiety which can be selectively “turned on” when the substrate compound and/or micelle is modified as described herein. The fluorescent moiety may comprise any entity that provides a fluorescent signal and that can be used in accordance with the methods and principles described herein. The fluorescence of the fluorescent moiety is quenched when the substrate compound is incorporated into the micelle. Activation of the trigger moiety initiates a spontaneous rearrangement that results in the fragmentation of the substrate compound to release either the fluorescent moiety or the hydrophobic moiety, thereby increasing the fluorescent signal produced by the fluorescent moiety.

Quenching of the fluorescent moiety within the micelle can be achieved in a variety of different ways. In one embodiment, the quenching effect may be achieved or caused by “self-quenching.” Self-quenching can occur when the substrate compounds comprising a micelle are present in the micelle at a concentration sufficient or molar ratio high enough to bring their fluorescent moieties in close enough proximity to one another such that their fluorescence signals are quenched. Release of the fluorescent moieties from the micelle reduces or abolishes the “self-quenching,” producing an increase in their fluorescence signals. As used herein, a fluorescent moiety is “released” or “removed” from a micelle if any molecule or molecular fragment that contains the fluorescent moiety is released or removed from the micelle.

For any given assay, the fluorescent moiety can be soluble or insoluble. For example, in some embodiments the fluorescent moiety is soluble under conditions of the assay so as to facilitate removal of the released fluorescent moiety from the micelle into the assay medium. In other embodiments, provided that self-quenching does not occur, the fluorescent moiety is insoluble under conditions of the assay so that the fluorescent moiety can precipitate out of solution and localize at the site at which it was produced, thereby producing an increase in the fluorescent signal as compared to the signal observed in solution.

The quenching effect may also be achieved or caused by other moieties comprising the micelle. These moieties are referred to as “quenching moieties,” regardless of the mechanism by which the quenching is achieved. Such quenching moieties and quenching molecules are described in more detail, below. By modifying the quenching moieties to reduce or eliminate their quenching effects, or by removing the fluorescent moiety from proximity of the quenching moieties, the fluorescence of the fluorescent moiety can be substantially restored. Any mechanism that is capable of causing quenching or changes in fluorescence properties may be used in the micelles and methods described herein.

The degree of quenching achieved within the micelle is not critical for success, provided that it is measurable by the detection system being used. As will be appreciated, higher degrees of quenching are desirable, because the greater the quenching effect, the lower the background fluorescence prior to removal of the quenching effect. In theory, a quenching effect of 100%, which corresponds to complete suppression of a measurable fluorescence signal, would be ideal. In practice, any measurable amount will suffice. The amount and/or molar percentage of substrate compound and optional quenching molecule in a micelle necessary to provide a desired degree of quenching in the micelle may vary depending upon, among other factors, the choice of the fluorescent moiety. The amount and/or molar percentage of any substrate compound (or mixture of substrate compounds) and optional quenching molecule (or mixture of optional quenching molecules) comprising a substrate compound-containing micelle in order to obtain a sufficient degree of quenching can be determined empirically.

Typically, the fluorescent moiety of the substrate compound comprises a fluorescent dye that in turn comprises a resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, such as xanthenes, rhodamines, fluoresceins, cyanines, phthalocyanines, squaraines, bodipy dyes, coumarins, oxazines, and carbopyronines.

In some embodiments, the fluorescent moiety comprises a xanthene dye. Generally, xanthene dyes are characterized by three main features: (1) a parent xanthene ring; (2) an exocyclic hydroxyl or amine substituent; and (3) an exocyclic oxo or imminium substituent. The exocyclic substituents are typically positioned at the C3 and C6 carbons of the parent xanthene ring, although “extended” xanthenes in which the parent xanthene ring comprises a benzo group fused to either or both of the C5/C6 and C3/C4 carbons are also known. In these extended xanthenes, the characteristic exocyclic substituents are positioned at the corresponding positions of the extended xanthene ring. Thus, as used herein, a “xanthene dye” generally comprises one of the following parent rings:

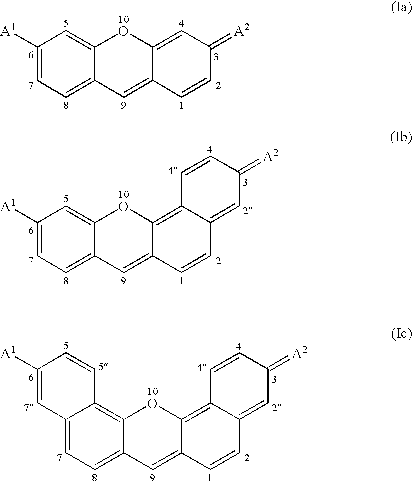

In the parent rings depicted above, A¹ is OH or NH₂ and A² is O or NH₂⁺. When A¹ is OH and A² is O, the parent ring is a fluorescein-type xanthene ring. When A¹ is NH₂ and A² is NH₂⁺, the parent ring is a rhodamine-type xanthene ring. When A¹ is NH₂ and A² is O, the parent ring is a rhodol-type xanthene ring.

One or both of nitrogens of A¹ and A² (when present) and/or one or more of the carbon atoms at positions C1, C2, C2″, C4, C4″, C5, C5″, C7″, C7 and C8 can be independently substituted with a wide variety of the same or different substituents. In one embodiment, typical substituents comprise, but are not limited to, —X, —R^{a}, —OR^{a}, —SR^{a}, —NR^{a}R^{a}, perhalo (C₁-C₆) alkyl, —CX₃, —CF₃, —CN, —OCN, —SCN, —NCO, —NCS, —NO, —NO₂, —N₃, —S(O)₂O—, —S(O)₂OH, —S(O)₂R^{a}, —C(O)R, —C(O)X, —C(S)R^{a}, —C(S)X, —C(O)OR^{a}, —C(O)O⁻, —C(S)OR^{a}, —C(O)SR^{a}, —C(S)SR^{a}, —C(O)NR^{a}R^{a}, —C(S)NR^{a}R^{a} and —C(NR)NR^{a}R^{a}, where each X is independently a halogen (preferably —F or —Cl) and each R^{a} is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, 5-20 membered heteroaryl, 6-26 membered heteroarylalkyl, 5-20 membered heteroaryl-heteroaryl, carboxyl, acetyl, sulfonyl, sulfinyl, sulfone, phosphate, or phosphonate. Generally, substituents which do not tend to completely quench the fluorescence of the parent ring are preferred, but in some embodiments quenching substituents may be desirable. Substituents that tend to quench fluorescence of parent xanthene rings are electron-withdrawing groups, such as —NO₂, —Br and —I.

The C1 and C2 substituents and/or the C7 and C8 substituents can be taken together to form substituted or unsubstituted buta[1,3]dieno or (C₅-C₂₀) aryleno bridges. For purposes of illustration, exemplary parent xanthene rings including unsubstituted benzo bridges fused to the C1/C2 and C7/C8 carbons are illustrated below:

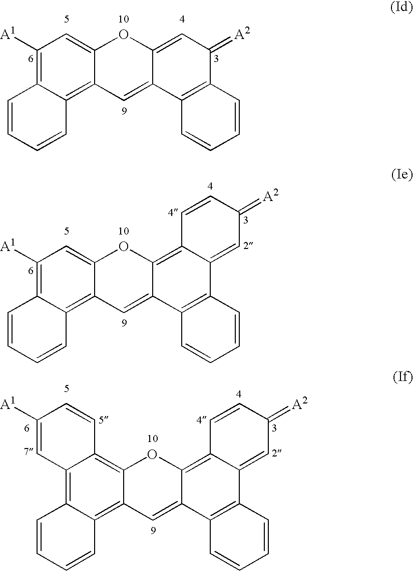

The benzo or aryleno bridges may be substituted at one or more positions with a variety of different substituent groups, such as the substituent groups previously described above for carbons C1-C8 in structures (Ia)-(Ic), supra. In embodiments including a plurality of substituents, the substituents may all be the same, or some or all of the substituents can differ from one another.

When A¹ is NH₂ and/or A² is NH₂⁺, the nitrogen atoms may be included in one or two bridges involving adjacent carbon atom(s). The bridging groups may be the same or different, and are typically selected from (C₁-C₁₂) alkyldiyl, (C₁-C₁₂) alkyleno, 2-12 membered heteroalkyldiyl and/or 2-12 membered heteroalkyleno bridges. Non-limiting exemplary parent rings that comprise bridges involving the exocyclic nitrogens are illustrated below:

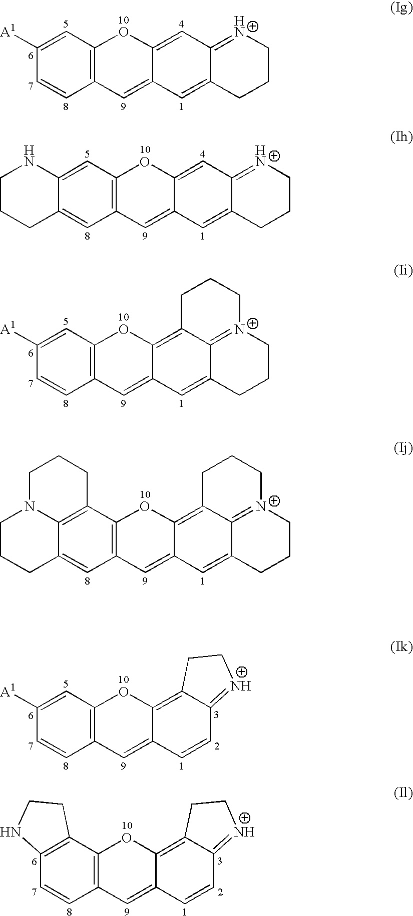

The parent ring may also comprise a substituent at the C9 position. In some embodiments, the C9 substituent is selected from acetylene, lower (e.g., from 1 to 6 carbon atoms) alkanyl, lower alkenyl, cyano, aryl, phenyl, heteroaryl, electron-rich heteroaryl and substituted forms of any of the preceding groups. In embodiments in which the parent ring comprises benzo or aryleno bridges fused to the C1/C2 and C7/C8 positions, such as, for example, rings (Id), (Ie) and (If) illustrated above, the C9 carbon is preferably unsubstituted.

In some embodiments, the C9 substituent is a substituted or unsubstituted phenyl ring such that the xanthene dye comprises one of the following structures:

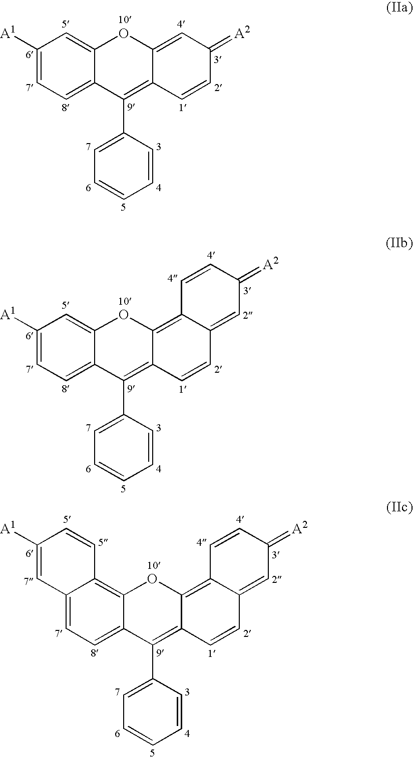

The carbons at positions 3, 4, 5, 6 and 7 may be substituted with a variety of different substituent groups, such as the substituent groups previously described for carbons C1-C8. In some embodiments, the carbon at position C3 is substituted with a carboxyl (—COOH) or sulfuric acid (—SO₃H) group, or an anion thereof. Dyes of formulae (IIa), (IIb) and (IIc) in which A¹ is OH and A² is O are referred to herein as fluorescein dyes; dyes of formulae (IIa), (IIb) and (IIc) in which A¹ is NH₂ and A² is NH₂⁺ are referred to herein as rhodamine dyes; and dyes of formulae (IIa), (IIb) and (IIc) in which A¹ is OH and A² is NH₂⁺ (or in which A¹ is NH₂ and A² is O) are referred to herein as rhodol dyes.

As highlighted by the above structures, when xanthene rings (or extended xanthene rings) are included in fluorescein, rhodamine and rhodol dyes, their carbon atoms are numbered differently. Specifically, their carbon atom numberings include primes. Although the above numbering systems for fluorescein, rhodamine and rhodol dyes are provided for convenience, it is to be understood that other numbering systems may be employed, and that they are not intended to be limiting. It is also to be understood that while one isomeric form of the dyes are illustrated, they may exist in other isomeric forms, including, by way of example and not limitation, other tautomeric forms or geometric forms. As a specific example, carboxy rhodamine and fluorescein dyes may exist in a lactone form.

In some embodiments, the fluorescent moiety comprises a rhodamine dye. Exemplary suitable rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichloro-tetramethylrhodamine (dTAMRA). Additional suitable rhodamine dyes include, for example, those described in U.S. Pat. Nos. 6,248,884, 6,111,116, 6,080,852, 6,051,719, 6,025,505, 6,017,712, 5,936,087, 5,847,162, 5,840,999, 5,750,409, 5,366,860, 5,231,191, and 5,227,487; PCT Publications WO 97/36960 and WO 99/27020; Lee et al., NUCL. ACIDS RES. 20:2471-2483 (1992), Arden-Jacob, NEUE LANWELLIGE XANTHEN-FARBSTOFFE FÜR FLUORESZENZSONDEN UND FARBSTOFF LASER, Verlag Shaker, Germany (1993), Sauer et al., J. FLUORESCENCE 5:247-261 (1995), Lee et al., NUCL. ACIDS RES. 25:2816-2822 (1997), and Rosenblum et al., NUCL. ACIDS RES. 25:4500-4504 (1997). A particularly preferred subset of rhodamine dyes are 4,7,-dichlororhodamines. In one embodiment, the fluorescent moiety comprises a 4,7-dichloro-orthocarboxyrhodamine dye.

In some embodiments, the fluorescent moiety comprises a fluorescein dye. Exemplary suitable fluorescein include, but are not limited to, fluorescein dyes described in U.S. Pat. Nos. 6,008,379, 5,840,999, 5,750,409, 5,654,442, 5,188,934, 5,066,580, 4,933,471, 4,481,136 and 4,439,356; PCT Publication WO 99/16832, and EPO Publication 050684. A preferred subset of fluorescein dyes are 4,7-dichlorofluoresceins. Other preferred fluorescein dyes include, but are not limited to, 5-carboxyfluorescein (5-FAM) and 6-carboxyfluorescein (6-FAM). In one embodiment, the fluorescein moiety comprises a 4,7-dichloro-orthocarboxyfluorescein dye.

In some embodiments, the fluorescent moiety can include a cyanine, a phthalocyanine, a squaraine, or a bodipy dye, such as those described in the following references and the references cited therein: U.S. Pat. Nos. 6,080,868, 6,005,113, 5,945,526, 5,863,753, 5,863,727, 5,800,996, and 5,436,134; and PCT Publication WO 96/04405.

In some embodiments, the fluorescent moiety can comprise a network of dyes that operate cooperatively with one another such as, for example by FRET or another mechanism, to provide large Stoke's shifts. Such dye networks typically comprise a fluorescence donor moiety and a fluorescence acceptor moiety, and may comprise additional moieties that act as both fluorescence acceptors and donors. The fluorescence donor and acceptor moieties can comprise any of the previously described dyes, provided that dyes are selected that can act cooperatively with one another. In a specific embodiment, the fluorescent moiety comprises a fluorescence donor moiety which comprises a fluorescein dye and a fluorescence acceptor moiety which comprises a fluorescein or rhodamine dye. Non-limiting examples of suitable dye pairs or networks are described in U.S. Pat. Nos. 6,399,392, 6,232,075, 5,863,727, and 5,800,996.

The substrate compound also comprises a trigger moiety that can be activated by a specified trigger agent. Any means of activating the trigger moiety may be used, provided that the means used to activate the trigger moiety is capable of producing a detectable change (e.g., an increase) in fluorescence. Preferably, the specified trigger agent is substantially active at the interface between the micelle and the assay medium. Selection of a particular means of activation, and hence trigger moiety, may depend, in part, on the particular fragmentation reaction, as well as on other factors.

In some embodiments, activation is based upon cleavage of the trigger moiety. In these embodiments, the trigger moiety comprises a cleavage site that is cleavable by a chemical reagent or cleaving enzyme. As a specific example, the trigger moiety can comprise a cleavage site that is cleavable by a lipase, an esterase, a phosphatase, a glycosidase, a protease, a nuclease or a catalytic antibody. The trigger moiety can further comprise additional residues and/or features that facilitate the specificity, affinity and/or kinetics of the cleaving enzyme. Depending upon the requirements of the particular cleaving enzyme, such cleaving enzyme “recognition moieties” can comprise the cleavage site or, alternatively, the cleavage site may be external to the recognition moiety. For example, certain endonucleases cleave at positions that are upstream or downstream of the region of the nucleic acid molecule bound by the endonuclease.

The chemical composition of the trigger moiety will depend upon, among other factors, the requirements of the cleaving enzyme. For example, if the cleaving enzyme is a protease, the trigger moiety can comprise a peptide (or analog thereof) recognized and cleaved by the particular protease. If the cleaving enzyme is a nuclease, the trigger moiety can comprise an oligonucleotide (or analog thereof) recognized and cleaved by a particular nuclease. If the cleaving enzyme is glycosidase, the trigger moiety can comprise a carbohydrate recognized and cleaved by a particular glycosidase.

Sequences and structures recognized and cleaved by the various different types of cleaving enzymes are well known. Any of these sequences and structures can comprise the trigger moiety. Although the cleavage can be sequence specific, in some embodiments it can be non-specific. For example, the cleavage can be achieved through the use of a non-sequence specific nuclease, such as, for example, an RNase.

Structures recognized and cleaved by glycosidases are also well known (see, e.g., Florent, et al., J. MED. CHEM. 41:3572-3581 (1998), Ghosh, et al., TETRAHEDRON LETTERS 41:4871-4874 (2000), Michel, et al., ATTA-UR-RAHMAN (ED) 21:157-180 (2000), and Leu, et al., J. MED. CHEM. 42:3623-3628 (1999)). Specific examples of substrate compounds comprising trigger moieties cleavable by glycosidases are described in more detail below.

Structures recognized and cleaved by lipases and esterases are also well known (see, e.g., Ohwada, et al., BIOORG. MED. CHEM. LETT. 12:2775-2780 (2002), Sauerbrei, et al., ANGEW. CHEM. INT. ED. 37:1143-1146 (1998), Greenwald, et al., J. MED. CHEM. LETT. 43:475-487 (2000), Dillon, et al., BIOORG. MED. CHEM. LETT. 14:1653-1656 (1996), and Greenwald, et al., J. MED. CHEM. 47:726-734 (2004)). Specific examples of substrate compounds comprising trigger moieties cleavable by lipases and esterases are described in more detail below. In embodiments utilizing lipases as the specified trigger agent, it will be understood that the hydrophobic moiety does not comprise any cleavage sites for the lipase trigger agent.

Structures recognized and cleaved by proteases/proteolytic enzymes are also well known (see, e.g., Niculescu-Duvaz, et al., J. MED. CHEM. 41:5297-5309 (1998), Niculescu-Duvaz, et al., J. MED. CHEM. 42:2485-2489 (1999), Greenwald, et al., J. MED. CHEM. 42:3657-3667 (1999), de Groot, et al., BIOORG. MED. CHEM. LETT. 12:2371-2376 (2002), Dubowchik, et al., BIOCONJUGATE CHEM. 13:855-869 (2002), and de Groot, et al., J. ORG. CHEM. 66:8815-8830 (2001)). Specific examples of substrate compounds comprising trigger moieties cleavable by protease plasmin, trypsin, and carboxypeptidase G2 are described in more detail below.

Structures recognized and cleaved by catalytic antibodies are also well known (see, e.g, Gopin, et al., ANGEW. CHEM. INT. ED. 42:327-332 (2003), Dinaut, et al., CHEM. COMMUN. 1386-1387 (2001)). Specific examples of substrate compounds comprising trigger moieties cleavable by catalytic enzymes are described in more detail below.

In some embodiments, cleavage of the trigger moiety by a trigger agent can initiate fragmentation of the substrate compound directly without the formation of an intermediate compound. For example, cleavage of the trigger moiety by a glycosidase can result in the direct formation of a π electron-donor moiety that initiates a spontaneous reaction resulting in the fragmentation of the substrate compound.

In other embodiments, cleavage of the trigger moiety by the specified trigger agent can initiate fragmentation of the substrate compound indirectly via formation of an intermediate compound. In these embodiments, the intermediate compound generates a π electron-donor moiety that initiates a spontaneous reaction resulting in fragmentation of the substrate compound. For example, the trigger moiety can comprise an aromatic nitro or azide group that can be reduced, thereby generating a π electron-donor moiety that is capable of initiating fragmentation of the substrate compound and release of the hydrophobic moiety or the fluorescent moiety.

Fragmentation of the substrate compound following cleavage of the trigger moiety by the corresponding cleaving enzyme can release the fluorescent moiety from the micelle, reducing or eliminating quenching and producing a measurable increase in fluorescence.

In other embodiments, the trigger moiety also serves as the linker moiety. In these embodiments, cleavage of the trigger moiety by a specified trigger agent also results in fragmentation of the substrate compound and release of the hydrophobic moiety or the fluorescent moiety.

In other embodiments, formation of a π electron-donor moiety utilizes the reduction of chemical groups, such as aromatic nitro or azide moieties, connected to the linker moiety. Reduction of the chemical group generates a π electron-donor moiety that can initiate a spontaneous rearrangement reaction, resulting in the fragmentation of the linker, thereby promoting the release of the fluorescent moiety from the micelle. The release of the fluorescent moiety from the micelle produces a measurable increase in the fluorescence of the fluorescent moiety.

The hydrophobic moiety, fluorescent moiety, and trigger moiety are connected to the linker moiety in any way that permits them to perform their respective functions. In some embodiments, the hydrophobic moiety and the fluorescent moiety are each, independently of the other, directly connected to the linker moiety. In other embodiments, the hydrophobic moiety and the fluorescent moiety are each, independently of the other, indirectly connected to the linker moiety via one or more optional linkages. The optional linkages can comprise a leaving group, which upon fragmentation of the substrate compound is released from the backbone of the linker, along with the moiety that is attached to it. For example, in some embodiments, the fluorescent moiety can be attached to the backbone of the linker moiety via a linkage comprising a leaving group, while the hydrophobic moiety can be attached to the backbone of the linker moiety via a stable linkage, e.g., a linkage that does not dissociate from the backbone of the linker following the fragmentation reaction.

Likewise, the trigger moiety can be directly connected to the π electron-donor moiety, or indirectly connected via one or more optional linkages. Typically, linkages used to attach the trigger moiety to the π electron donor moiety are used to modulate the enzyme activity towards the trigger agent. For example, if cleavage of the trigger moiety is susceptible to steric hindrance, e.g., β-galactosidase, linkages could be used to distance the trigger moiety from the linker moiety. Alternatively, if the trigger agent is too reactive, e.g., an esterase or phosphatase, addition of the appropriate linkage can increase steric hindrance.

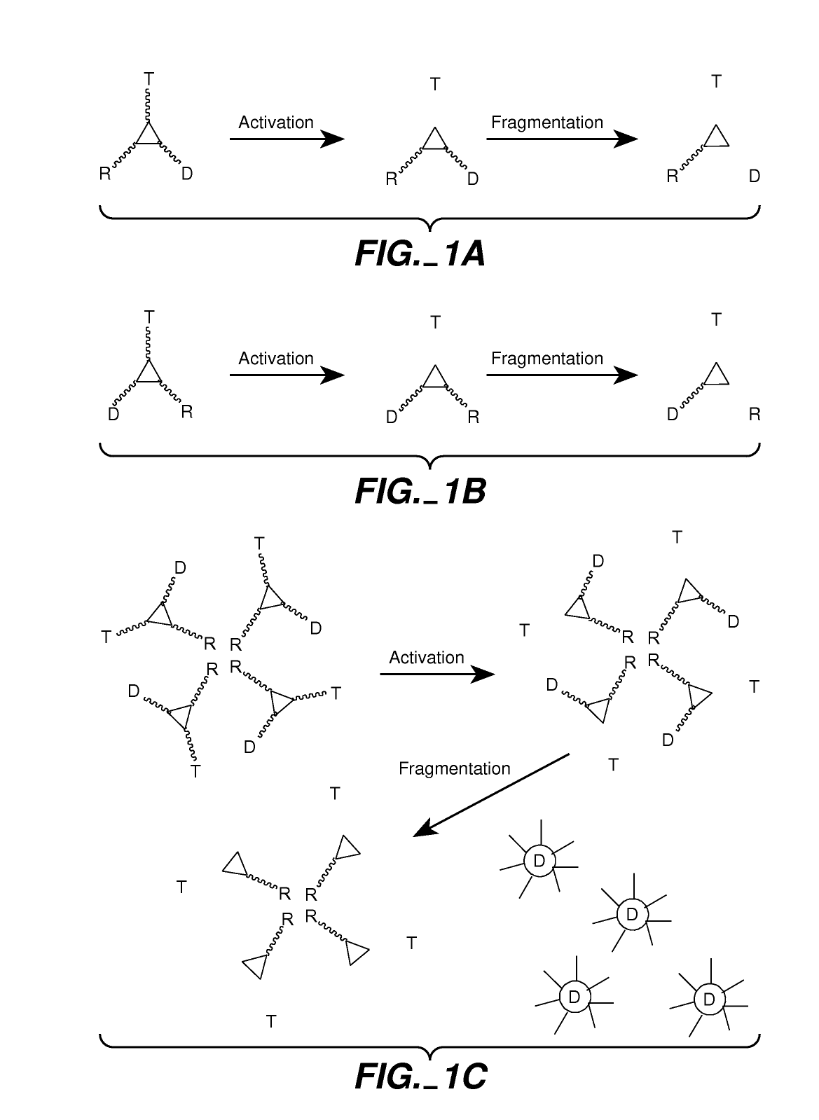

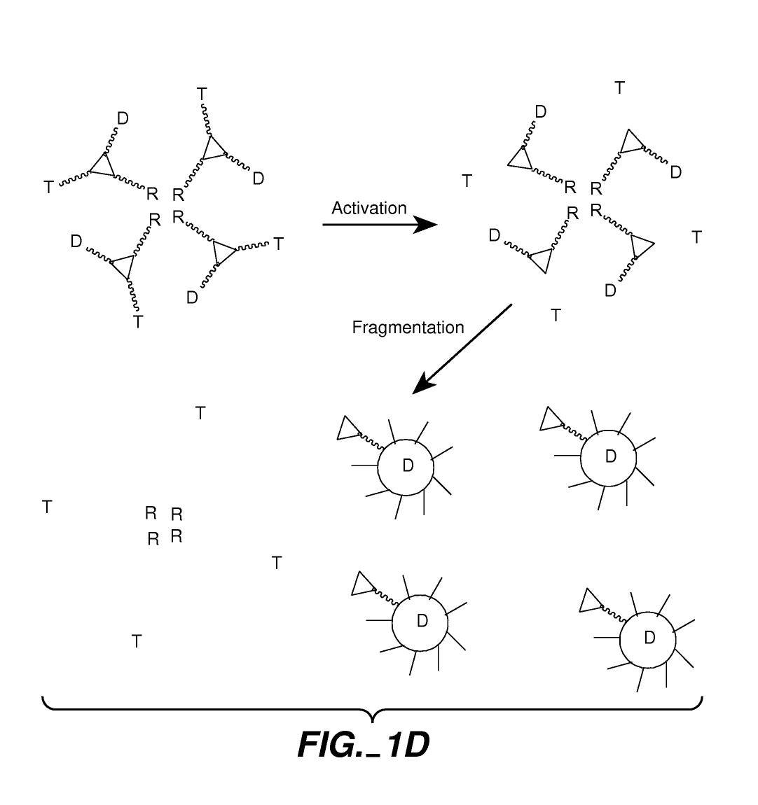

FIGS. 1A and 1B illustrate exemplary embodiments of a substrate compound comprising a trigger moiety T, a fluorescent moiety D, and a hydrophobic moiety, R, each of which, are independently of the other, attached to the backbone of a linker moiety. As illustrated in FIGS. 1A and 1B, the backbone of the linker moiety comprises three sites for the attachment of other molecules. Generally, the attachment site for the trigger moiety includes the π electron-donor moiety. The other two sites can be used for the attachment of optional linkage groups that can be used interchangeably for the attachment of the fluorescent moiety and the hydrophobic moiety. As will be appreciated by a person of skill in the art, the linker moiety illustrated in FIGS. 1A and 1B is merely exemplary, and linker moieties with two, three or more sites for the attachment of T, R, D, and optional substituent groups can be used in the compositions and methods described herein.

As illustrated in FIGS. 1A and 1B, fluorescent moiety D comprises a fluorescent dye. However, any reporter moiety that is operative in accordance with the various compositions and methods described herein can be used in place of D to detect the presence and/or quantity of a molecule of interest.

As illustrated in FIGS. 1A and 1B, R can comprise any of the hydrophobic groups described above. For example, R can comprise saturated or unsaturated alkyl chains, which may be same or different. In other embodiments, R can comprise a phospholipid comprising at least two hydrophobic moieties, e.g., R¹ and R², as described above.

As illustrated in FIGS. 1A and 1B, T can comprise any of the trigger moieties outlined above, which when activated by a specified trigger agent are capable of initiating a spontaneous rearrangement reaction that promotes fragmentation of the substrate compound and release of the fluorescent moiety or the hydrophobic moiety. For example, T can comprise a cleavage site that is recognized and cleaved by a cleaving enzyme, such as a lipase, an esterase, a phosphatase, a glycosidase, a carboxypeptidase or a catalytic antibody. Alternatively, T can comprise an aromatic nitro or azide group that can be reduced, thereby generating a π electron-donor group that is capable of initiating fragmentation of the substrate compound and release of the hydrophobic moiety or the fluorescent moiety.

In the exemplary embodiments illustrated in FIG. 1A or 1B, fluorescent moiety D or hydrophobic moiety R is released from the backbone of the linker moiety via a spontaneous rearrangement reaction. Spontaneous rearrangement reactions capable of fragmenting the substrate compound and releasing D or R include 1,4-, bis 1,4-, 1,6-, mono 1,8-, and bis 1,8-elimination reactions, and ring closure mechanisms, such as trimethyl lock lactonization reactions and intramolecular cyclization reactions.

In the exemplary embodiment illustrated in FIG. 1A, release of fluorescent moiety D is initiated by activation of T by a specified trigger agent. In some embodiments, T comprises a cleavage site for a cleaving enzyme. Activation is initiated when the cleaving enzyme recognizes and cleaves T at the cleavage site, thereby generating a π electron-donor moiety that is capable of initiating a spontaneous rearrangement reaction that results in the cleavage of T from the backbone of the linker moiety. Subsequent rearrangement(s) result in the fragmentation of the linker and release of D.

In other embodiments, T comprises a reactive nitro or azide group. In these embodiments, a π electron-donor moiety is generated when the nitro or azide group is reduced. Reduction of the nitro or azide group generates a π electron-donor moiety, e.g., —NH—, that is capable of initiating a spontaneous rearrangement reaction that results in the cleavage of T from the backbone of the linker. Subsequent rearrangement(s) result in the fragmentation of the linker and release of D.

In the exemplary embodiment illustrated in FIG. 1B, hydrophobic moiety R is released from the backbone of the linker as described above. In this embodiment, D remains attached to the backbone of the linker.

While the basis for increased fluorescence is not certain, and the inventors do not wish to be bound to a particular theory, it is contemplated that the fluorescent substrates described herein are capable of forming micelles in the reaction mixture due to the hydrophobic moiety, so that the fluorescent moieties quench each other due to their close proximity. Micelle formation can be particularly favored when the substrate is neutrally charged or has a small negative or small positive net charge, so that micelle formation is not prevented by mutual charge repulsion. The putative micelles may be in equilibrium with monomolecular, unassociated species in solution, but the micellar form is the predominant form.

As illustrated in FIG. 1C, if the fluorescent moiety is released by the fragmentation reaction, the “free” fluorescent moiety fluoresces brightly since it remains relatively free from other fluorescent substrate molecules in the solution.

As illustrated in FIG. 1D, if the hydrophobic moiety is released by the fragmentation reaction, it remains associated with the micelle, while the backbone of the linker comprising the fluorescent moiety is released from the micelle. As illustrated in FIG. 1D, the “free” fluorescent moiety fluoresces brightly since it remains relatively free from other fluorescent substrate molecules in the solution.

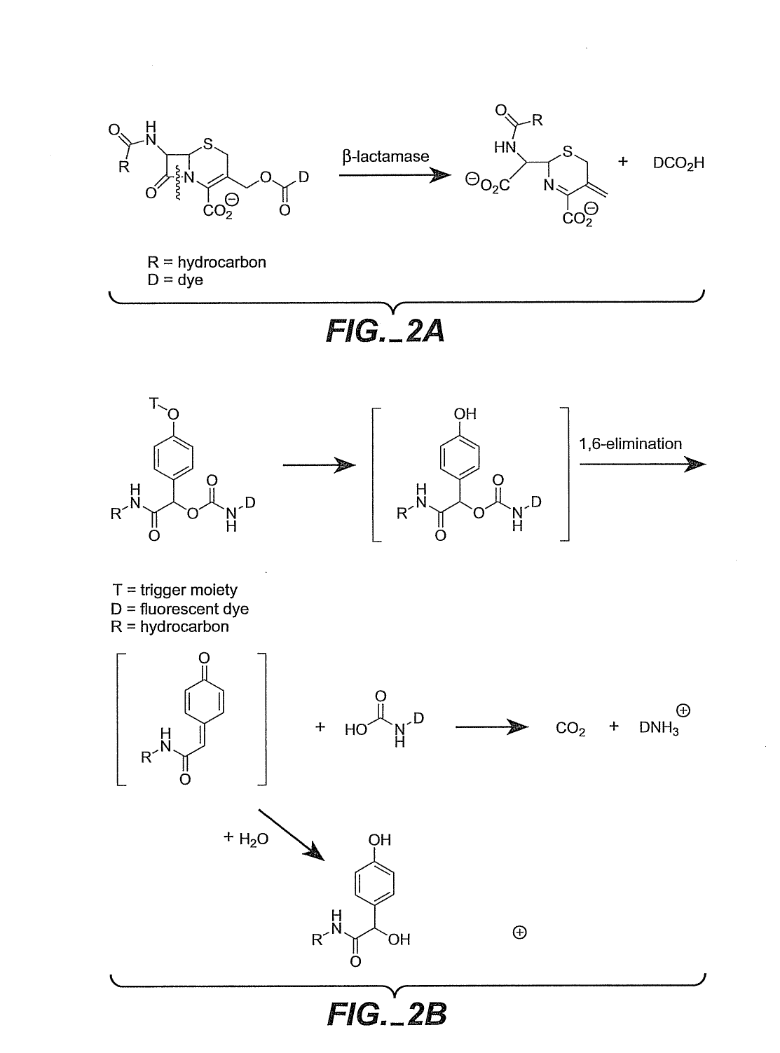

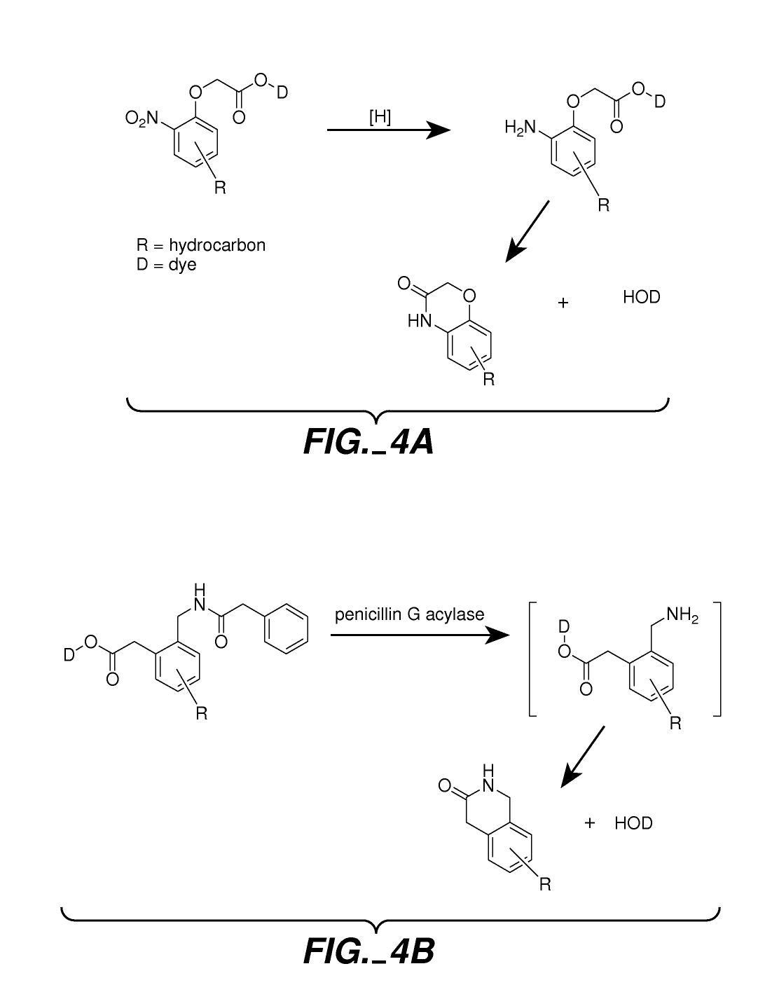

FIG. 2A illustrates an exemplary embodiment of a substrate compound in which the linker moiety also serves as the trigger moiety. In the embodiment illustrated in FIG. 4, the linker moiety comprises a beta-lactam molecule that undergoes a spontaneous self-elimination reaction to release D when cleaved by beta lactamase.

### 6.4 Substrate Compounds that Fragment Via an Elimination Reaction

In some embodiments, the substrate compound comprises a linker moiety that fragments via an elimination reaction. Various elimination reactions, such as 1,4-, 1,6- and 1,8-elimination reactions have been used in the design of prodrugs and can be easily adapted for use in the compositions and methods described herein. See, e.g., WO 02/083180, Gopin, et al., ANGEW. CHEM. INT. ED. 42:327-332 (2003), Niculescu-Duvaz, et al., J. MED. CHEM. 41:5297-5309 (1998), Florent, et al., J. MED. CHEM. 41:3572-3581 (1998), Niculescu-Duvaz, et al., J. MED. CHEM. 42:2485-2489 (1999), Greenwald, et al., J. MED. CHEM. 42:3657-3667 (1999), de Groot, et al., BIOORG. MED. CHEM. LETT. 12:2371-2376 (2002), Ghosh, et al., TETRAHEDRON LETTERS 41:4871-4874 (2000), Dubowchik, et al., BIOCONJUGATE CHEM. 13:855-869 (2002), Michel, et al., ATTA-UR-RAHMAN (ED) 21:157-180 (2000), Dinaut, et al., CHEM. COMMUN. 1386-1387 (2001), Ohwada, et al., BIOORG. MED. CHEM. LETT. 12:2775-2780 (2002), de Groot, et al., J. ORG. CHEM. 66:8815-8830 (2001), Leu, et al, J. MED. CHEM. 42:3623-3628 (1999), Sauerbrei, et al, ANGEW. CHEM. INT. ED. 37:1143-1146 (1998), Veinberg et al., BIOORG. MED. CHEM. LETT. 14:1007-1010 (2004), Greenwald, et al., BIOCONJUGATE CHEM. 14:395-403 (2003), and Lee et al., ANGEW. CHEM. INT. ED. 43:1675-1678 (2004).

The linker moiety comprises attachment sites for the attachment of the fluorescent moiety, hydrophobic moiety, trigger moiety, and one or more optional substituent groups. One of the attachment sites comprises a π electron-donor moiety that can be used for the attachment of the trigger moiety. The trigger moiety can be attached directly to the π electron-donor moiety, or indirectly to the π electron-donor moiety via one or more optional linkages For example, the trigger moiety can be attached to the backbone of the linker directly via a π electron-donor moiety, such as —O—, —S, or —NH—, or it can be attached indirectly to the backbone of the linker moiety via an optional linkage L, such as a —COO⁻—.

Other attachment sites comprise linkages for the attachment of the fluorescent moiety and the hydrophobic moiety. The fluorescent moiety and hydrophobic moiety can be attached to the same attachment site or to different attachment sites. Linkages useful for attaching the fluorescent moiety and the hydrophobic moiety include linkages having the general formula L¹ and L², wherein L¹ represents a linkage that is stable under the conditions of the assay, such that the linkage does not dissociate from the backbone of the linker moiety following the fragmentation reaction. L² represents a linkage comprising a leaving group. Examples of linkages suitable for use in the compositions and methods are described below.

In some embodiments, substrate compounds capable of fragmenting by an elimination reaction have the structure shown below:

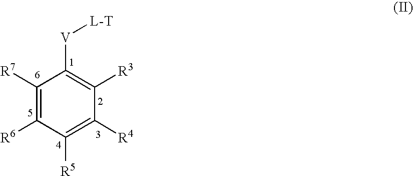

In structure II, “V” represents a π electron donor moiety, “L” represents an optional linkage group, “T” represents a trigger moiety, R³, R⁴, R⁵, R⁶, and R⁷ each independently comprise attachment sites for the attachment of the fluorescent moiety, the hydrophobic moiety and one or more optional substituent groups, “Y”.

In the exemplary substrate compound illustrated in Structure II, “V” can be O, NH, or S. “L” is an optional linkage group that can be used to attach the trigger moiety “T” to the backbone of the aromatic linker, such as those described below and in Table 2. Typically L is used to module the activity of the trigger agent. For example, if the activity of the trigger agent is susceptible to steric hindrance, an optional linkage can be used to “distance” the trigger moiety from the sterically crowded linker moiety. Alternatively, if the trigger agent is too reactive, an optional linkage can be used to increase the steric hindrance. Linkages suitable for modulating the enzyme activity are known to those of skill in the art, and include —COO⁻—.

Suitable trigger moieties include those that are cleaved by an enzyme or can be reduced under reducing conditions. Typically, the compositions described herein use trigger moieties that are cleaved by an enzyme. Examples of suitable “T” cleavage sites, cleaving enzymes, and optional linkage groups are provided in Table 2.

TABLE 2

| | Cleavage Site with | |
|---|---|---|
| Cleavage Site | Optional Linkage group | Cleaving Enzyme |
| 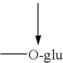 | 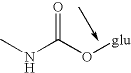 | β-glucuronidase |
| 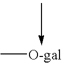 | 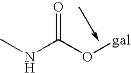 | β-galactosidase |
| 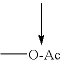 | | lipase/esterase |
| 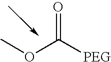 | 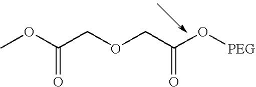 | lipase/esterase |
| 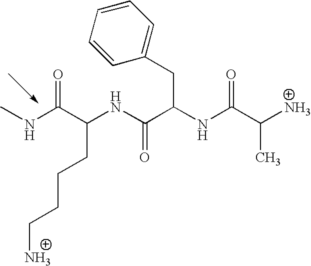 | | protease plasmin |
| 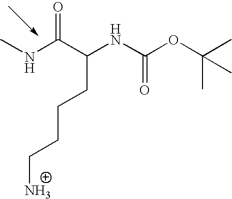 | | trypsin |
| 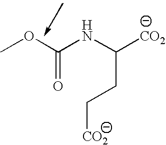 | | carboxypeptidaseG2 |
| 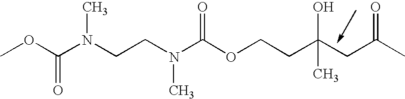 | | catalytic antibody |
| 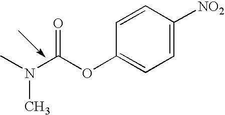 | | catalytic antibody |

(Glu and gal represent the carbohydrates glucoronide and galactose, respectively. The cleavage site is indicated by an arrow.)

The illustrated cleavage sites, cleavage sites with optional linkages and cleaving enzymes are merely exemplary trigger moieties and trigger agents. Any trigger moiety comprising a cleavage site suitable for cleavage by a cleavage enzyme that can be appropriately cleaved, leaving behind the π electron donor moiety could be used to provide an appropriate cleavage site. For example, a cleavage site comprising a phosphate group capable of being cleaved by a phosphatase could be used as trigger moiety and the corresponding phosphatase used as the specified trigger agent (see, e.g., Zhu, et al., BIOORG. MED. CHEM. LETT. 10:1121-1124 (2000), and Ueda, et al., BIOORG. MED. CHEM. LETT. 8:1761-1766 (1993)).

In other embodiments, T can comprise an aromatic nitro or azide group directly attached to the carbon atom at position C1 of the exemplary linker moieties illustrated in Structure II. Similar linker moieties are described in Damen, et al., for the delivery of prodrugs (Damen, et al., BIOORG. MED. CHEM. 10:71-77). Exemplary substrate compounds comprising an aromatic nitro or azide group are shown below:

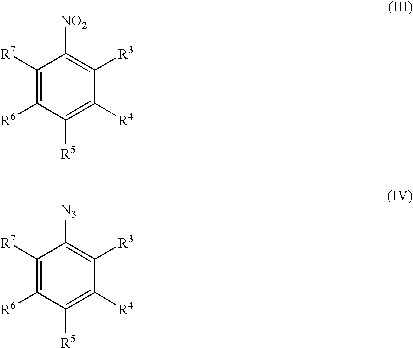

In the illustrated structures II-IV, R³, R⁴, R⁵, R⁶, and R⁷ are each independently the sites of attachment for the fluorescent moiety, the hydrophobic moiety and one or more optional substituent groups. In structures II, III, and IV, R³, R⁴, R⁵, R⁶, and R⁷ can be independently selected from:

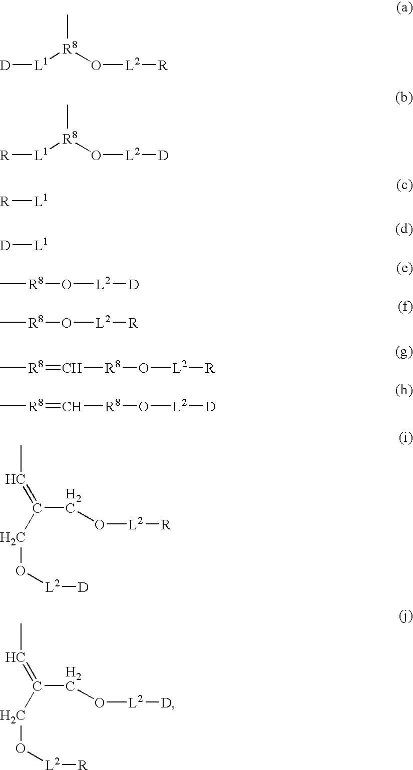

as well as from hydrogen, alkyl, aryl, cycloalkyl, heterocycloalkyl, heteroaryl, alkoxy, hydroxy, thiohydroxy, thioalkoxy, aryloxy, thiosaryloxy, amino, nitro, halo, trihalomethyl, cyano, C-amido, N-amido, imidazolyl, alkylpiperazinyl, morpholino, tetrazole, carboxy, carboxylate, sulfoxy, sulfonate, sulfonyl, sulfixy, suflinate, sulfinyl, phosphonooxy, or phosphate, or alternatively, at least two of R³, R⁴, R⁵, R⁶, and R⁷ can be connected to one another to form an aromatic or aliphatic cyclic structure;
wherein:
D is a fluorescent dye moiety as described herein;
R is a hydrophobic moiety as described herein;
R⁸ can be selected from the group consisting of CH, CR, CHR, and CR₂;
L¹ represents a stable linkage, including but not limited to an amide linkage, an —N—O— linkage, and a —N≡N— linkage
L² represents a linkage comprising a leaving group Z, and can be selected from the structures shown below:

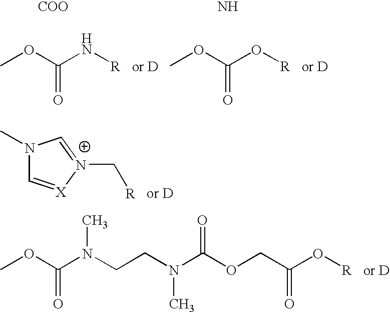

The fragmentable linker moieties illustrated in Structures II-IV comprising a benzyl backbone are merely exemplary linkers. Any molecule which is capable of fragmenting, and which comprises two or more “sites” suitable for attaching other molecule and moieties thereto, or that can be appropriately functionalized to attach other molecules and moieties thereto could be used to provide a divalent or higher order linker moiety. Although the “backbone” of the fragmentable linker moiety depicted in Structures II-IV is illustrated as an aryl compound comprising carbon and hydrogen atoms, the linker backbone need not be limited to carbon and hydrogen atoms. Thus, a linker backbone suitable for use in the compositions and methods described herein can include single, double, triple or aromatic carbon-carbon bonds, carbon-nitrogen bonds, nitrogen-nitrogen bonds, carbon-oxygen bonds, carbon-sulfur bonds and combinations thereof, and therefore can include substituents such as carbonyls, ethers, thioethers, carboxamides, sulfonamides, ureas, urethanes, hydrazines, etc. Moreover, the backbone of the linker moiety can comprise a mono or polycyclic aryl or an arylalkyl moiety.

In the exemplary substrate compounds of Structure II-IV, one or more optional “Y” substituents can be attached to R³, R⁴, R⁵, R⁶, and R⁷. The substituents may all be the same, or some or all of them may be different. Examples of suitable Y substituents groups, include but are not limited, —NO₂—, —CH₃—, —OCH₃—, —OR—, —Cl—, —F—, —NH₂—, —CO₂H—, and CH₂ CO₂NH₂—.

Skilled artisans will appreciate that the linkages discussed above for the attachment of the trigger moiety, the fluorescent moiety and the hydrophobic moiety are merely exemplary linkages. The trigger, hydrophobic and fluorescent moieties comprising the substrate compound can be linked to the backbone of the linker moiety via any linkage that is operative in the conditions under which the substrates will be used. Choosing a linkage having properties suitable for a particular application is within the capabilities of those having skill in the art. For example, the linkages on the linker moiety may all be the same, or some or all of them may be different.

Generally, linkages are selected that have different chemical substituents to facilitate the selective attachment of the fluorescent and hydrophobic moieties, to the linker moiety. The length and chemical composition of the linkage can be selectively varied. In some embodiments, the linkage can be selected to have specified properties. For example, the linkage can be hydrophobic in character, hydrophilic in character, long or short, rigid, semirigid or flexible, depending upon the particular application. The linkage can be optionally substituted with one or more substituents or one or more groups for the attachment of additional substituents, which may be the same or different, thereby providing a “polyvalent” capable of conjugating additional molecules or substances to the molecule. In certain embodiments, however, the linkage does not comprise such additional substituents.

A wide variety of linkages comprised of stable bonds that are suitable for use in the substrates described herein are known in the art, and include by way of example and not limitation, alkyldiyls, substituted alkyldiyls, alkylenos (e.g., alkanos), substituted alkylenos, heteroalkyldiyls, substituted heteroalkyldiyls, heteroalkylenos, substituted heteroalkylenos, acyclic heteroatomic bridges, aryldiyls, substituted aryldiyls, arylaryldiyls, substituted arylaryldiyls, arylalkyldiyls, substituted arylalkyldiyls, heteroaryldiyls, substituted heteroaryldiyls, heteroaryl-heteroaryl diyls, substituted heteroaryl-heteroaryl diyls, heteroarylalkyldiyls, substituted heteroarylalkyldiyls, heteroaryl-heteroalkyldiyls, substituted heteroaryl-heteroalkyldiyls, and the like. Thus, the linkage can include single, double, triple or aromatic carbon-carbon bonds, nitrogen-nitrogen bonds, carbon-nitrogen bonds, carbon-oxygen bonds, carbon-sulfur bonds and combinations of such bonds, and may therefore include functionalities such as carbonyls, ethers, thioethers, carboxamides, sulfonamides, ureas, urethanes, hydrazines, etc. In some embodiments, the linkage comprises from 1-20 non-hydrogen atoms selected from the group consisting of C, N, O, and S and is composed of any combination of ether, thioether, amine, ester, carboxamide, sulfonamides, hydrazide, aromatic and heteroaromatic groups.

Choosing a linkage having properties suitable for a particular application is within the capabilities of those having skill in the art. For example, where a rigid linkage is desired, it may comprise a rigid polypeptide such as polyproline, a rigid polyunsaturated alkyldiyl or an aryldiyl, biaryldiyl, arylarydiyl, arylalkyldiyl, heteroaryldiyl, biheteroaryldiyl, heteroarylalkyldiyl, heteroaryl-heteroaryldiyl, etc. Where a flexible linkage is desired, it may comprise a flexible polypeptide such as polyglycine or a flexible saturated alkanyldiyl or heteroalkanyldiyl. Hydrophilic linkages may comprise, for example, polyalcohols or polyethers such as polyalkyleneglycols, or other spacers. Hydrophobic linkages may comprise, for example, alkyldiyls or aryldiyls.

In some embodiments, the linkages are formed from pairs of complementary reactive groups capable of forming covalent linkages with one another. “Complementary” nucleophilic and electrophilic groups (or precursors thereof that can be suitably activated) useful for effecting linkage stable to biological and other assay conditions are well known. Examples of suitable complementary nucleophilic and electrophilic groups, as well as the resultant linkages formed therefrom, are provided in Table 3.

TABLE 3

| Electrophilic Group | Nucleophilic Group | Resultant Covalent Linkage |
|---|---|---|
| activated esters* | amines/anilines | Carboxamides |
| acyl azides** | amines/anilines | Carboxamides |
| acyl halides | amines/anilines | Carboxamides |
| acyl halides | alcohols/phenols | Esters |
| acyl nitriles | alcohols/phenols | Esters |
| acyl nitriles | amines/anilines | Carboxamides |
| Aldehydes | amines/anilines | Imines |
| aldehydes or ketones | hydrazines | Hydrazones |
| aldehydes or ketones | hydroxylamines | Oximes |
| alkyl halides | amines/anilines | alkyl amines |
| alkyl halides | carboxylic acids | Esters |
| alkyl halides | Thiols | Thioethers |
| alkyl halides | alcohols/phenols | Ethers |
| alkyl sulfonates | Thiols | Thioethers |
| alkyl sulfonates | carboxylic acids | Esters |
| alkyl sulfonates | alcohols/phenols | Esters |
| Anhydrides | alcohols/phenols | Esters |
| Anhydrides | amines/anilines | Caroboxamides |
| aryl halides | Thiols | Thiophenols |
| aryl halides | Amines | aryl amines |
| Aziridines | Thiols | Thioethers |
| Boronates | Glycols | boronate esters |
| carboxylic acids | amines/anilines | Carboxamides |
| carboxylic acids | alcohols | Esters |
| carboxylic acids | hydrazines | Hydrazides |
| Carbodiimides | carboxylic acids | N-acylureas or anhydrides |
| Diazoalkanes | carboxylic acids | Esters |
| Epoxides | Thiols | Thioethers |
| Haloacetamides | Thiols | Thioethers |
| Halotriazines | amines/anilines | Aminotriazines |
| Halotriazines | alcohols/phenols | triazinyl ethers |
| imido esters | amines/anilines | Amidines |
| Isocyanates | amines/anilines | Ureas |
| Isocyanates | alcohols/phenols | Urethanes |
| Isothiocyanates | amines/anilines | Thioureas |
| Maleimides | Thiols | Thioethers |
| Phosphoramidites | alcohols | phosphate esters |
| silyl halides | alcohols | silyl ethers |
| sulfonate esters | amines/anilines | alkyl amines |
| sulfonate esters | Thiols | Thioethers |
| sulfonate esters | carboxylic acids | Esters |
| sulfonate esters | alcohols | Esters |
| sulfonyl halides | amines/anilines | Sulfonamides |
| sulfonyl halides | Phenols/alcohols | sulfonate esters |
| diazonium salt | aryl | azo |

*Activated esters, as understood in the art, generally have the formula —C(O)Z, where Z is, a good leaving group (e.g., oxysuccinimidyl, oxysulfosuccinimidyl, 1-oxybenzotriazolyl, etc.).
**Acyl azides can rearrange to isocyanates.

FIG. 2B illustrates an exemplary embodiment of a substrate compound in which the substrate compound fragments via a 1,6-elimination reaction. In the embodiment illustrated in FIG. 2B, the substrate compound generally comprises a trigger moiety (represented by T), a fluorescent moiety (represented by D), a hydrophobic moiety (represented by R), and a linker moiety comprising a benzyl backbone. In the embodiment illustrated in FIG. 2B, the π electron-donor moiety attached to the carbon atom at position C1 of the benzyl backbone can comprise a reactive —O— group as shown, or a reactive —NH— or —S— group. In the embodiment illustrated in FIG. 2B, trigger moiety T is connected directly to the reactive —O— group. In other embodiments, T can be indirectly connected to the reactive —O— group via an additional linkage L, such as those described above.

In the embodiment illustrated in FIG. 2B, D and R are both attached to the benzyl linker at the C4 carbon via a CH group. In the embodiment illustrated in FIG. 2A, D is attached via a L² linkage, e.g., —O—C(O)—NH, and R is attached via a stable L¹ linkage, e.g., —C(O)—NH.

The addition of a specified trigger agent to the substrate compound illustrated in FIG. 2B initiates a 1,6-elimination reaction by removing T and generating a reactive hydroxy group at the C1 carbon of the benzyl backbone. The hydroxy group so generated spontaneously promotes the 1,6-elimination reaction resulting in the release of the HOCONHD moiety. Further rearrangement results in the release of CO₂ and DNH₃⁺. In the embodiment illustrated in FIG. 2B, R remains attached to the backbone of the benzyl linker moiety.

Exemplary benzyl linker structures that can be used for 1,4- and 1,6-elimination reactions are shown below in Table 4.

TABLE 4

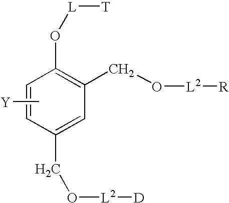

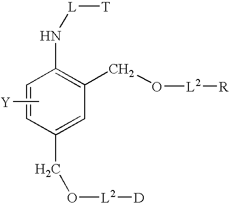

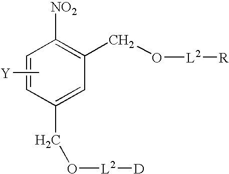

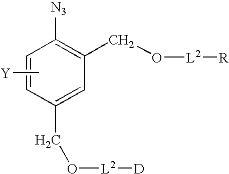

L and L² represent linkage groups as described above. L is an optional linkage depending on whether the activity of the trigger agent needs to be modulated. L² represents a linkage comprising a leaving group.

Y represents one or more optional substituent groups as described above, that can be attached at any site not used for the attachment of the fluorescent moiety or the hydrophobic moiety. For example if the fluorescent moiety is attached to the benzyl linker at the C4 carbon and the hydrophobic moiety is attached to the benzyl linker at the C2 position, then Y can be attached at the C3, C4 and/or C5 carbon atoms.

Exemplary embodiments of benzyl linker structures that can be used in 1,6-elimination reactions are illustrated below in Table 5.

TABLE 5

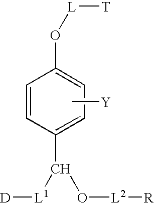

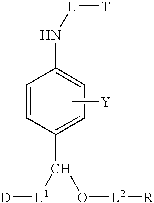

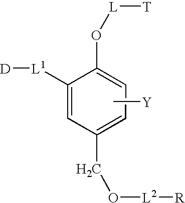

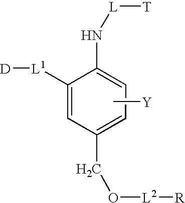

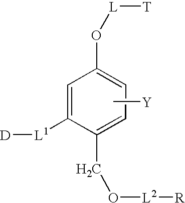

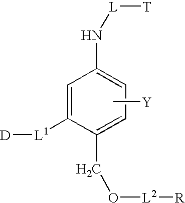

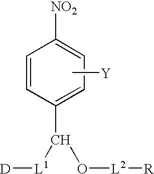

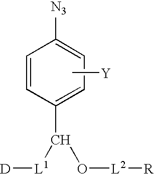

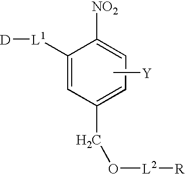

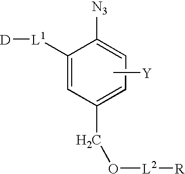

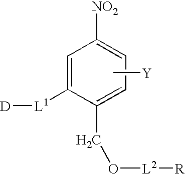

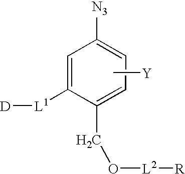

L, L¹, and L² represent linkage groups as described above. L is an optional linkage depending on whether the activity of the trigger agent needs to be modulated. L¹ represents a stable linkage, while L² represents a linkage comprising a leaving group. Although the above structures are illustrated with the hydrophobic moiety attached to the leaving group, similar structures can be designed in which the fluorescent moiety is attached to L².

Y represents one or more optional substituent groups as described above, that can be attached at any attachment site that is not used for the attachment of the fluorescent moiety or the hydrophobic moiety. For example, if both the hydrophobic moiety and the fluorescent moiety are attached to the C4 carbon atom, then Y can be attached at the C2, C3 and/or C5 carbon atoms.

Exemplary embodiments of benzyl linker structures that can be used in 1,4-elimination reactions are illustrated below in Table 6.

TABLE 6

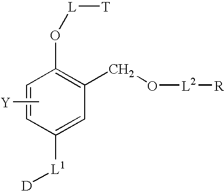

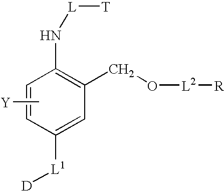

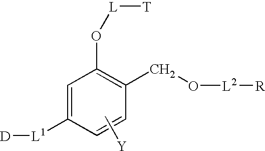

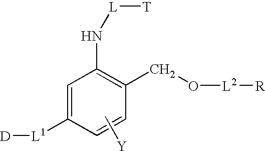

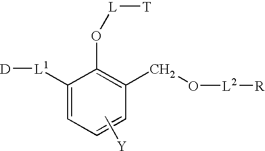

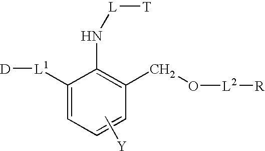

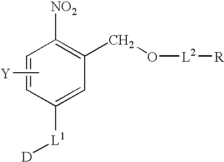

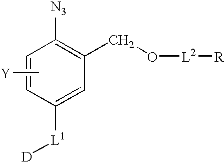

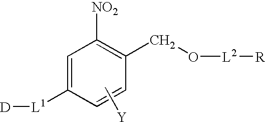

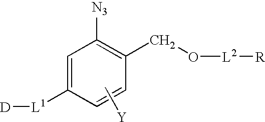

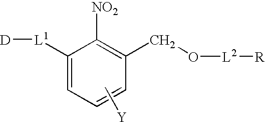

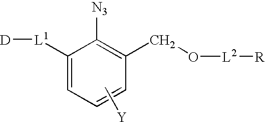

L, L¹, and L² represent linkage groups as described above. L is an optional linkage depending on whether the activity of the trigger agent needs to be modulated. L¹ represents a stable linkage, while L² represents a linkage comprising a leaving group. Although the above structures are illustrated with the hydrophobic moiety attached to the leaving group, similar structures can be designed in which the fluorescent moiety is attached to L².

Y represents one or more optional substituent groups as described above, that can be attached at any attachment site that is not used for the attachment of the fluorescent moiety or the hydrophobic moiety. For example, if the hydrophobic moiety is attached at the C2 carbon atom and the fluorescent moiety is attached to the C5 carbon atom, then Y can be attached at the C3 and/or C4 carbon atoms.

In other embodiments, benzyl linkers for bis 1,4-elimination reactions can be used in the compositions and methods described herein. Exemplary benzyl linker structures for bis 1,4-elimination reactions are shown in Table 7.

TABLE 7

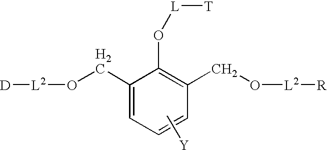

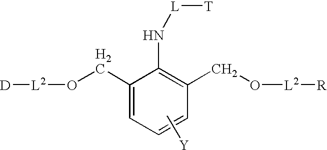

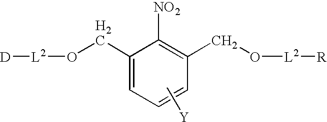

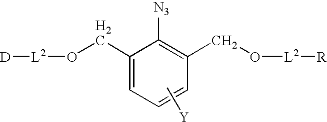

L and L² represent linkage groups as described above. L is an optional linkage depending on whether the activity of the trigger agent needs to be modulated. L² represents a linkage comprising a leaving group.

Y represents one or more optional substituent groups as described above, that can be attached at any attachment site that is not used for the attachment of the fluorescent moiety or the hydrophobic moiety. For example, if the hydrophobic moiety is attached at the C2 carbon atom and the fluorescent moiety is attached to the C6 carbon atom, then Y can be attached at the C3, C4 and/or C5 carbon atoms.

Exemplary embodiments of benzyl linker structures that can be used in 1,8-elimination reactions are illustrated below in Table 8.

TABLE 8

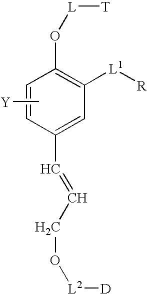

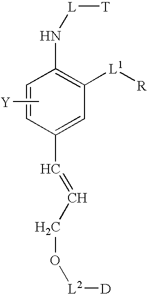

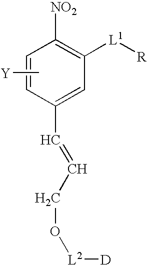

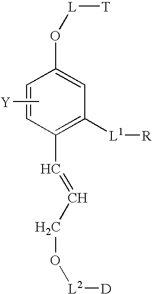

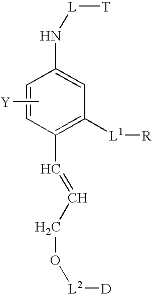

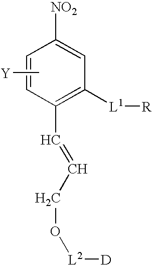

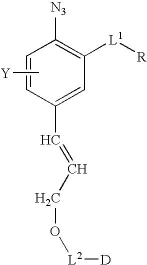

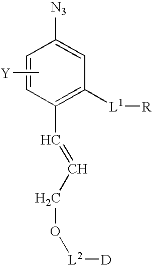

L, L¹, and L² represent linkage groups as described above. L is an optional linkage depending on whether the activity of the trigger agent needs to be modulated. L¹ represents a stable linkage, while L² represents a linkage comprising a leaving group. Although the above structures are illustrated with the fluorescent moiety attached to the leaving group, similar structures can be designed in which the hydrophobic moiety is attached to L². Y represents one or more optional substituent groups as described above, that can be attached at any attachment site that is not used for the attachment of the fluorescent moiety or the hydrophobic moiety. For example, if the hydrophobic moiety is attached to the C3 carbon atom and the fluorescent moiety is attached to the C4 carbon atom, then Y can be attached to the C2, C5 and/or C6 carbon atoms.

In other embodiments, benzyl linkers for bis 1,8-elimination reactions can be used in the compositions and methods described herein. Exemplary benzyl linker structures for bis 1,8-elimination reactions are shown in Table 9.

TABLE 9

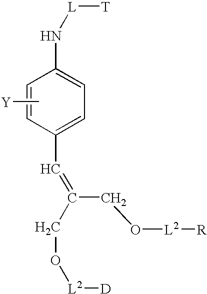

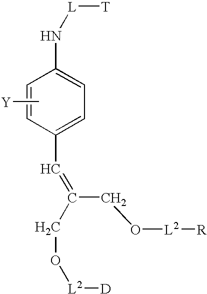

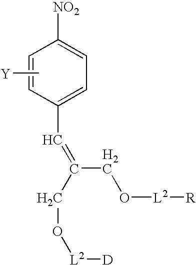

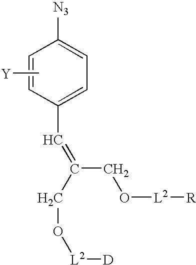

L and L² represent linkage groups as described above. L is an optional linkage depending on whether the activity of the trigger agent needs to be modulated. L² represents a linkage comprising a leaving group.

Y represents one or more optional substituent groups as described above, that can be attached at any attachment site that is not used for the attachment of the fluorescent moiety or the hydrophobic moiety. For example, if the hydrophobic moiety and the fluorescent moiety are attached to the C4 carbon atom, then Y can be attached to the C2, C3, C5, and/or C6 carbon atoms.

Skilled artisans will appreciate that while the substrate compounds illustrated in Tables 4-9 are not exemplified with specific trigger moieties, functional groups, hydrophobic moieties, or fluorescent moieties any one of the various moieties described herein can be used with the generalized linker structures illustrated in Tables 4-9. Moreover, virtually any type of chemical linkage(s) that is stable to the assay conditions and that permit the various moieties to perform their respective functions could be used. Additionally, the various illustrated features can be readily “mixed and matched” to provide other specific embodiments of exemplary substrate compounds.

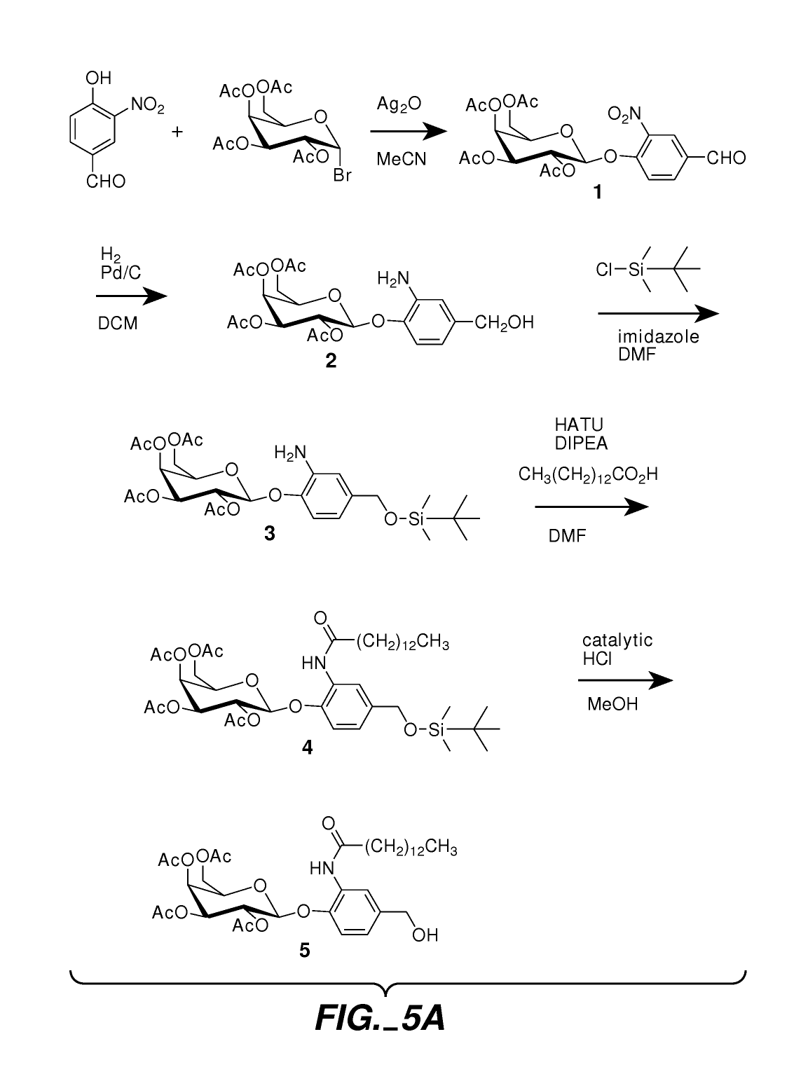

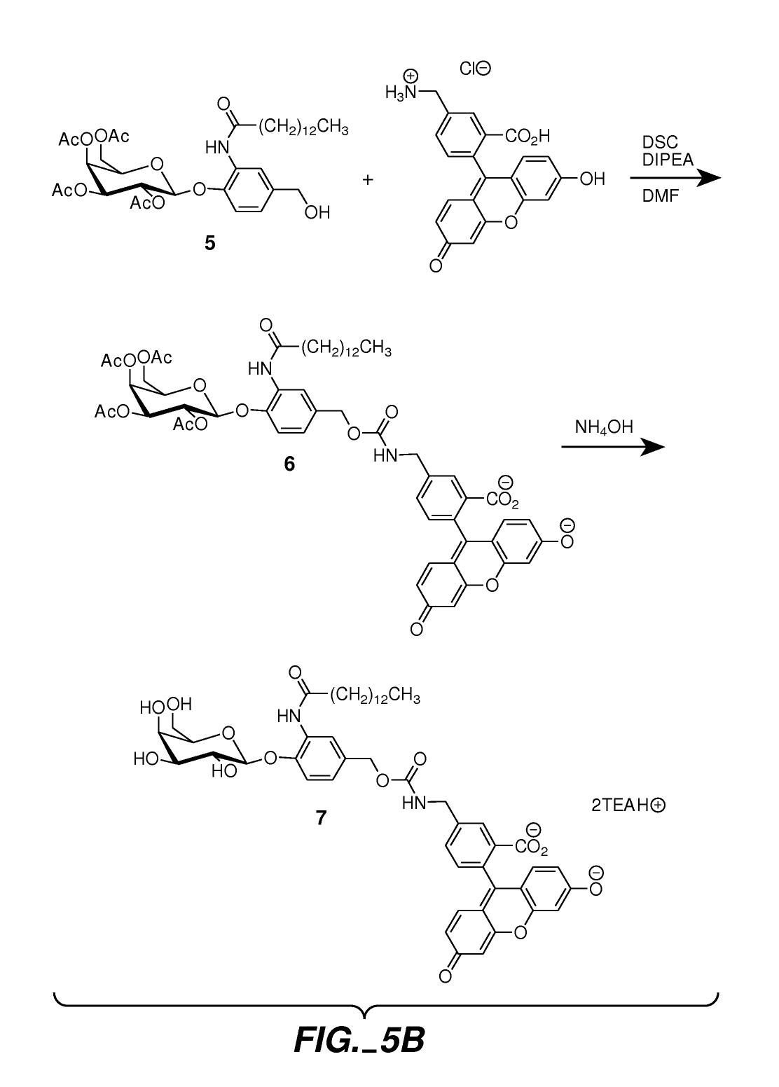

Substrate compounds comprising benzyl linkers capable of undergoing a 1-4- or a 1-6 elimination reaction can be synthesized according to the scheme illustrated in FIGS. 5A-5B. Referring to FIG. 5A, bromo 2,3,4,6-tetra-O-acetyl-α-D-galactopyranoside and 4-hydroxy-3-nitrobenzaldehyde are reacted in the presence of silver oxide to yield compound 1. Compound 1 can be dissolved in dichloromethane (DCM) and converted by catalytic hydrogenation to yield compound 2. Compound 2 can be dissolved in dry dimethylformamide (DMF) and reacted with imidazole and tert-butyldimethylsilyl chloride to yield compound 3. Compound 3 can be reacted with myristic acid, N,N-diisopropylethylamine (DIPEA) and N—[(dimethylamino)-1H-1,2,3-triazolo[4,5-b]pyridin-1-ylmethylene]-N-methylmethanaminium hexafluorophosphate N-oxide (HATU) to yield compound 4. Compound 4 can be dissolved in a solution of HCl in MeOH, followed by a neutralization reaction with NaHCO₃ to yield compound 5. Compound 5 can be reacted with 5-(aminomethyl)fluorescein hydrochloride in the presence of N,N′-disuccinimidyl carbonate (DSC) and DIPEA to yield compound 6. Ammonium hydroxide can be added to compound 6 and the resulting reaction mixture purified by reverse phase HPLC to obtain compound 7.

Trigger moieties that can be attached to the backbone of the linker moiety, as exemplified in FIGS. 5A-5B, supra, can be prepared using conventional methods. For example, trigger moieties comprising a peptide sequence can be prepared using automated synthesizers on a solid support (Perkin J. Am. Chem. Soc. 85:2149-2154 (1963)) by any of the known methods, e.g. Fmoc or BOC (e.g., Atherton, J. Chem. Soc. 538-546 (1981); Fmoc Solid Phase Peptide Synthesis. A Practical Approach, Chan, Weng C. and White, Peter D., eds., Oxford University Press, New York, 2000). Synthetically, polypeptides may be formed by a condensation reaction between the α-carbon carboxyl group of one amino acid and the amino group of another amino acid. Activated amino acids are coupled onto a growing chain of amino acids, with appropriate coupling reagents. Polypeptides can be synthesized with amino acid monomer units where the α-amino group was protected with Fmoc (fluorenylmethoxycarbonyl). Alternatively, the BOC method of peptide synthesis can be practiced to prepare the peptide conjugates described herein.

Amino acids with reactive side-chains can be further protected with appropriate protecting groups. Amino groups on lysine side-chains to be labelled can be protected with an Mtt protecting group, selectively removable with about 5% trifluoroacetic acid in dichloromethane. A large number of different protecting group strategies can be employed to efficiently prepare polypeptides.

Exemplary solid supports include polyethyleneoxy/polystyrene graft copolymer supports (TentaGel, Rapp Polymere GmbH, Tubingen, Germany) and a low-cross link, high-swelling Merrifield-type polystyrene supports with an acid-cleavable linker (Applied Biosystems), although others can be used as well.

Polypeptides are typically synthesized on commercially available synthesizers at scales ranging from 3 to 50 μmoles. The Fmoc group is removed from the terminus of the peptide chain with a solution of piperidine in dimethylformamide (DMF), typically 30% piperidine, requiring several minutes for deprotection to be completed. The amino acid monomer, coupling agent, and activator are delivered into the synthesis chamber or column, with agitation by vortexing or shaking. Typically, the coupling agent is HBTU, and the activator is 1-hydroxybenzotriazole (HOBt). The coupling solution also may contain diisopropylethylamine or another organic base, to adjust the pH to an optimal level for rapid and efficient coupling.

Peptides may alternatively be prepared on chlorotrityl polystyrene resin by typical solid-phase peptide synthesis methods with a Model 433A Peptide Synthesizer (Applied Biosystems, Foster City, Calif.) and Fmoc/HBTU chemistry (Fields, (1990) Int. J. Peptide Protein Res. 35:161-214). The crude protected peptide on resin may be cleaved with 1% trifluoroacetic acid (TFA) in methylene chloride for about 10 minutes. The filtrate is immediately raised to pH 7.6 with an organic amine base, e.g. 4-dimethylaminopyridine. After evaporating the volatile reagents, a crude protected peptide is obtained. If desired, the crude peptide can be labelled with additional groups.

Following synthesis, the peptide on the solid support (resin) is deprotected and cleaved from the support. Deprotection and cleavage may be performed in any order, depending on the protecting groups, the linkage between the peptide and the support, and, if labeling is desired, the labeling strategy. After cleavage and deprotection, peptides may be desalted by gel filtration, precipitation, or other means, and analyzed. Typical analytical methods useful for the peptides and peptide conjugates comprising the substrate compounds include mass spectroscopy, absorption spectroscopy, HPLC, and Edman degradation sequencing. The peptides and peptide conjugates may be purified by reverse-phase HPLC, gel filtration, electrophoresis, or dialysis.

Hydrophobic moieties that can be attached to the backbone of the linker moiety, as exemplified in FIGS. 5A-5B, supra, are available commercially. The synthesis of phospholipids is described in PHOSPHOLIPIDS HANDBOOK (G. Cevc, ed., Marcel Dekker (1993)), BIOCONJUGATE TECHNIQUES (G. Hermanson, Academic Press (1996)), and Subramanian et al., ARKIVOC VII:116-125 (2002), for example.

Fluorescent dyes that can be used to prepare the substrate compounds described herein, can be prepared synthetically using conventional methods or purchased commercially (e.g. Sigma-Aldrich and/or Molecular Probes). Non-limiting examples of methods that can be used to synthesize suitably reactive fluorescein and/or rhodamine dyes can be found in the various patents and publications discussed above in connection with the fluorescent moiety. Non-limiting examples of suitably reactive fluorescent dyes that are commercially available from Molecular Probes (Eugene, Oreg.) are provided in Table 10, below:

TABLE 10

| Catalog | |
|---|---|
| Number | Product Name |
| C-20050 | 5-carboxyfluorescein-bis-(5-carboxymethoxy- |
| | 2-nitrobenzyl) ether, -alanine-carboxamide, |
| | succinimidyl ester (CMNB-caged |
| | carboxyfluorescein, SE) |
| C-2210 | 5-carboxyfluorescein, succinimidyl ester |
| | (5-FAM, SE) |
| C-1311 | 5-(and-6)-carboxyfluorescein, succinimidyl |
| | ester (5(6)-FAM, SE) |
| D-16 | 5-(4,6-dichlorotriazinyl) aminofluorescein |
| | (5-DTAF) |
| F-6106 | 6-(fluorescein-5-carboxamido)hexanoic acid, |
| | succinimidyl ester (5-SFX) |
| F-2182 | 6-(fluorescein-5-(and-6)-carboxamido) hexanoic |
| | acid, succinimidyl ester (5(6)-SFX) |
| F-6129 | 6-(fluorescein-5-(and-6)-carboxamido) hexanoic |
| | acid, succinimidyl ester (5(6)-SFX) |
| F-6130 | fluorescein-5-EX, succinimidyl ester |
| F-143 | fluorescein-5-isothiocyanate (FITC ‘Isomer I’) |
| F-1906 | fluorescein-5-isothiocyanate (FITC ‘Isomer I’) |
| F-1907 | fluorescein-5-isothiocyanate (FITC ‘Isomer I’) |
| F-144 | fluorescein-6-isothiocyanate (FITC ‘Isomer II’) |
| A-1353 | 5-(aminomethyl)fluorescein |
| T-353 | Texas Red ® sulfonyl chloride |
| T-1905 | Texas Red ® sulfonyl chloride |
| T-10125 | Texas Red ®-X, STP ester, sodium salt |
| T-6134 | Texas Red ®-X, succinimidyl ester |
| T-20175 | Texas Red ®-X, succinimidyl ester |

The syntheses of exemplary substrate compound(s) that fragment via a 1-4- or a 1-6 elimination reaction according to the Scheme illustrated in FIGS. 5A-5B, is discussed in more detail in the Examples Section. Methods for the synthesis of additional substrate compounds capable of fragmenting via a 1,4- or a 1,6-elimination reaction are provided in the Examples.

### 6.5 Substrate Compounds that Fragment Via Intramolecular Cyclization

In some embodiments, the substrate compound comprises a linker moiety that fragments via a ring closure mechanism. Exemplary ring closure mechanisms include trimethyl lock lactonization reactions (see, e.g., Greenwald, et al., J. MED. CHEM. LETT. 43:475-487 (2000), Cheruvallath, et al., BIOORG. MED. CHEM. LETT.: 281-284 (2003), Zhu, et al., BIOORG. MED. CHEM. LETT. 10:1121-1124 (2000), Dillon, et al., BIOORG. MED. CHEM. LETT. 14:1653-1656 (1996), Ueda, et al., BIOORG. MED. CHEM. LETT. 8:1761-1766 (1993)) and intramolecular cyclization reactions using safety catch linkers (see, e.g., Greenwald, et al., J. MED. CHEM. 47:726-734 (2004).

Exemplary substrate compounds capable of fragmenting by a trimethyl lock lactonization reaction have the structure shown below:

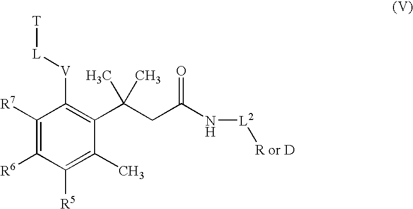

In the embodiment illustrated in Structure V, the backbone of the linker moiety is a phenyl group comprising two, three or more sites that can be used to attach the trigger moiety, hydrophobic moiety and fluorescent moiety to the backbone of the linker moiety. Although the backbone of the linker moiety is illustrated as a phenyl, the linker backbone need not be limited to carbon and hydrogen atoms. For example, the linker backbone could include heteroaryl compounds comprising carbon-nitrogen bonds, nitrogen-nitrogen bonds, carbon-oxygen bond, carbon-sulfur bonds and combinations thereof.

As illustrated in Structure V, R⁵, R⁶, and R⁷ can comprise an optional substituent group “Y”, L¹-R or L¹-D. L, L¹, and L² represent linkage groups as described above. The selection of the various combinations of substituents, will depend in part, on whether the hydrophobic moiety or fluorescent moiety is attached to L². For example, if the fluorescent moiety is attached to L², then any one R⁵, R⁶, and R⁷ can comprise L¹-D and, if desired, optional Y groups, provided that they are connected in a way that permits them to perform their respective functions and in a manner that does not interfere with the fragmentation of the substrate compound and release of the fluorescent moiety. Similarly, if the hydrophobic moiety is attached to L², then any one R⁵, R⁶, and R⁷ can comprise L¹-D and, if desired, optional Y groups, provided that they are connected in a way that permits them to perform their respective functions and in a manner that does not interfere with the fragmentation of the substrate compound and release of the hydrophobic moiety.

A wide variety of optional Y substituents that are suitable for use with linker moieties that fragment via a ring closure method are known in the art, and include by way of example and not limitation —H—, —CH₃—, and —(CH₂)ₙCO₂H—.

The trigger moiety (represented by T) is attached to the C1 carbon of the phenyl linker backbone via a reactive —O—. In other embodiments, the trigger moiety can be attached to the C1 carbon via a reactive —NH— group. In addition, an optional linkage L can be used to link T to the reactive —O— or —NH— moiety, or to facilitate the specificity, affinity and/or kinetics of the specified trigger agent. Examples of suitable trigger moieties and corresponding trigger agents are provided in Table 11 below.

TABLE 11

| Trigger Moiety | Trigger Agent |
|---|---|
| PO₃H⁻ | Phosphatase |
| 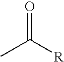 | Lipase |
| 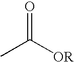 | Esterase |
| 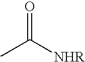 | Protease |

As will be appreciated by a person skilled in the art, the illustrated trigger moieties and trigger agents provided in Table 11 are merely exemplary trigger moieties and trigger agents. Any trigger moiety comprising a cleavage site suitable for cleavage by a cleavage enzyme and that can be appropriately cleaved to provide a reactive —O— or —NH— group could be used to provide a trigger moiety. In some embodiments, an optional linkage can be used to modulate the activity of the trigger agent. For example, a cleavage site comprising a carbohydrate moiety capable of being cleaved and an optional linkage could be used as the trigger moiety and the corresponding glycosidase used as the specified trigger agent.

In the exemplary substrate compound illustrated in Structure V, a linkage group, i.e., —CH(CH₃)₂CH₂CO-Z capable of undergoing a cyclization reaction is attached to the carbon atom at position C2 of the phenyl backbone. This linkage group serves as point of attachment for a leaving group Z to which can be attached the fluorescent moiety or the hydrophobic moiety. Suitable Z moieties include —NH— and —O.

Additional linkages groups can be used for the attachment of the hydrophobic moiety or fluorescent moiety to carbon atoms at positions C3, C4, C5 or C6. Suitable linkage groups include those discussed above for embodiments in which the linker moiety fragments by an elimination reaction.

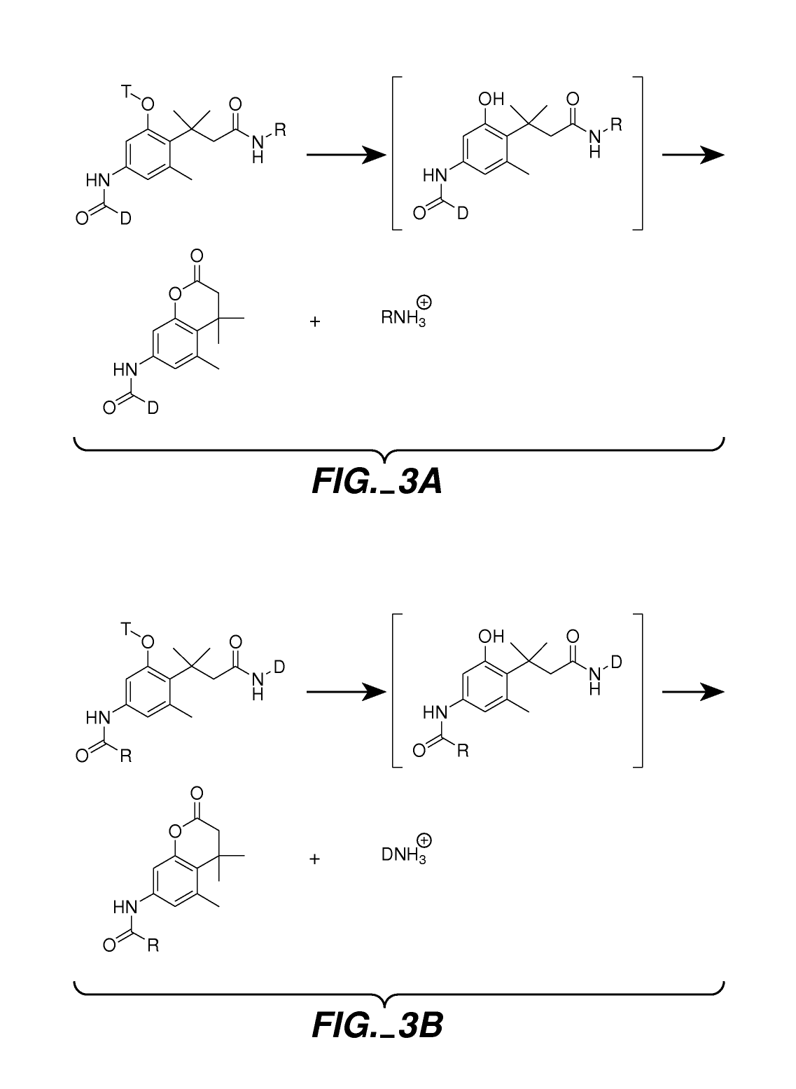

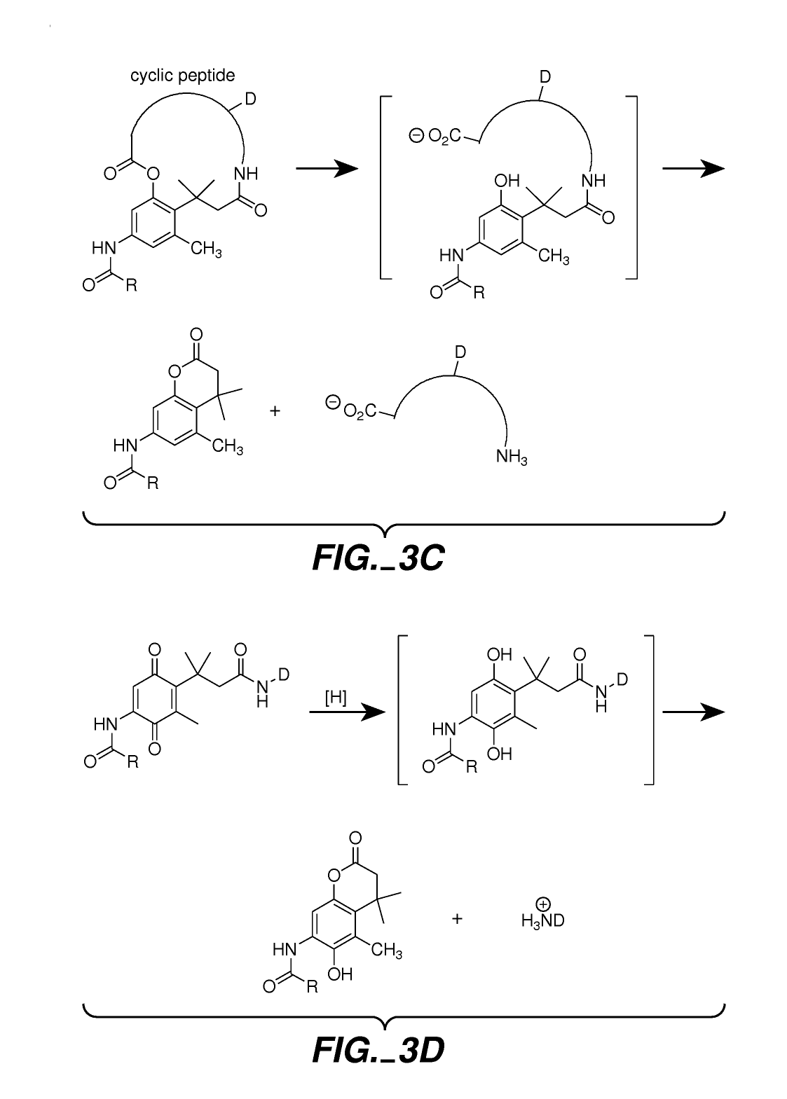

In the exemplary substrate compound illustrated in FIG. 3A, the hydrophobic moiety (represented by R) is attached to a linkage group that is capable of cyclizing following activation of the trigger moiety by a specified trigger agent. Cyclization of the illustrated linkage group results in the release of the R from the backbone of the linker moiety. As illustrated in FIG. 3B, the fluorescent moiety (represented by D) is attached to a linkage that participates in the cyclization reaction. Thus, in the embodiment illustrated in FIG. 3B, D is released from the backbone of the linker moiety.

An exemplary substrate compound fragmented via a trimethyl lock lactonization reaction is illustrated in FIG. 3C. In the exemplary substrate illustrated in FIG. 3C, T comprises a cleavage site for an esterase, Z comprises a cyclic peptide leaving group to which D is connected, Y comprises a methyl group attached to carbon atom C3, and the hydrophobic moiety is attached to C4 via a —CONH— linkage group. Cleavage of T by an esterase initiates the trimethyl lock lactonization reaction, thereby releasing D.

In the exemplary substrate compound embodiment illustrated in FIG. 3D, fragmentation via a trimethyl lock lactonization reaction is activated under reducing conditions that convert the nitro group to a reactive —NH— group. The reactive —NH— group then initiates a lactonization reaction that results in the release of D.

Substrate compounds capable of fragmenting by a ring closure mechanism utilizing a safety catch linker have the structure shown below:

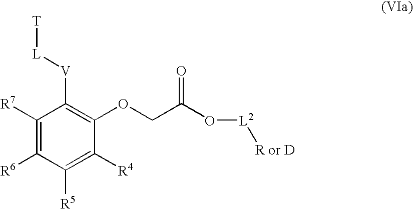

In the embodiment illustrated in Structure VIa, the backbone of the linker moiety is a phenyl group comprising two, three or more sites that can be used to attach the trigger moiety, hydrophobic moiety and fluorescent moiety to the backbone of the linker. Although the backbone of the linker moiety is illustrated as a phenyl, the backbone of the linker moiety need not be limited to carbon and hydrogen atoms. For example, the backbone of the linker could include heteroaryl compounds comprising carbon-nitrogen bonds, nitrogen-nitrogen bonds, carbon-oxygen bond, carbon-sulfur bonds and combinations thereof.

In the exemplary embodiment illustrated in Structure VIa, the trigger moiety (represented by T) is attached to the carbon atom at position C1 of the phenyl backbone. As described above, T comprises a π electron-donor moiety (i.e. V) to which is attached, directly or indirectly via an optional linkage L, a cleavage site for a cleaving enzyme. In other embodiments, e.g., Structure VIb, T can comprise an aromatic nitro or azide group that can be reduced to generate a π electron-donor moiety.

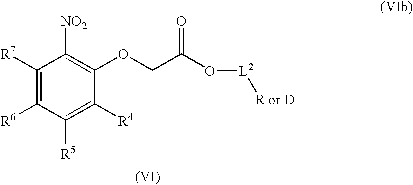

As illustrated in Structure Via or VIb, R⁴, R⁵, R⁶ and R⁷ can comprise the hydrophobic moiety, the fluorescent moiety and one or more optional substituent groups (not shown). The location of the fluorescent moiety or the hydrophobic moiety, will depend in part, on whether the hydrophobic moiety or fluorescent moiety is attached to the L² linkage group. For example, if the fluorescent moiety is attached to the L² linkage group, then any one of R⁴, R⁵, R⁶ and R⁷ can comprise L¹-R and, if desired, optional Y groups, provided that L¹-R and Y are connected in a way that permits them to perform their respective functions and in a manner that does not interfere with the fragmentation of the substrate compound and release of the fluorescent moiety. Similarly, if the hydrophobic moiety is attached to the L² linkage group, then any one of R⁴, R⁵, R⁶ and R⁷ can comprise L¹-D and, if desired, optional Y groups, provided that L¹-D and Y are connected in a way that permits them to perform their respective functions and in a manner that does not interfere with the fragmentation of the substrate compound and release of the hydrophobic moiety.

In the exemplary substrate compound illustrated in FIG. 4A, fragmentation via a ring closure reaction using a “safety catch linker” is activated by a reductive environment that converts the nitro group to a reactive —NH— group. In the exemplary embodiment illustrated in FIG. 4A, the electronic cascade reaction initiates cleavage of the ester moiety, ring closure, and release of D.

In the exemplary substrate compound illustrated in FIG. 4B, fragmentation via a ring closure reaction using a “safety catch linker” is activated by a cleaving enzyme, i.e. pencillin G acylase. Cleavage by pencillin G acylase generates a reactive —NH₂— group that initiates a ring closure reaction that results in the release of D.

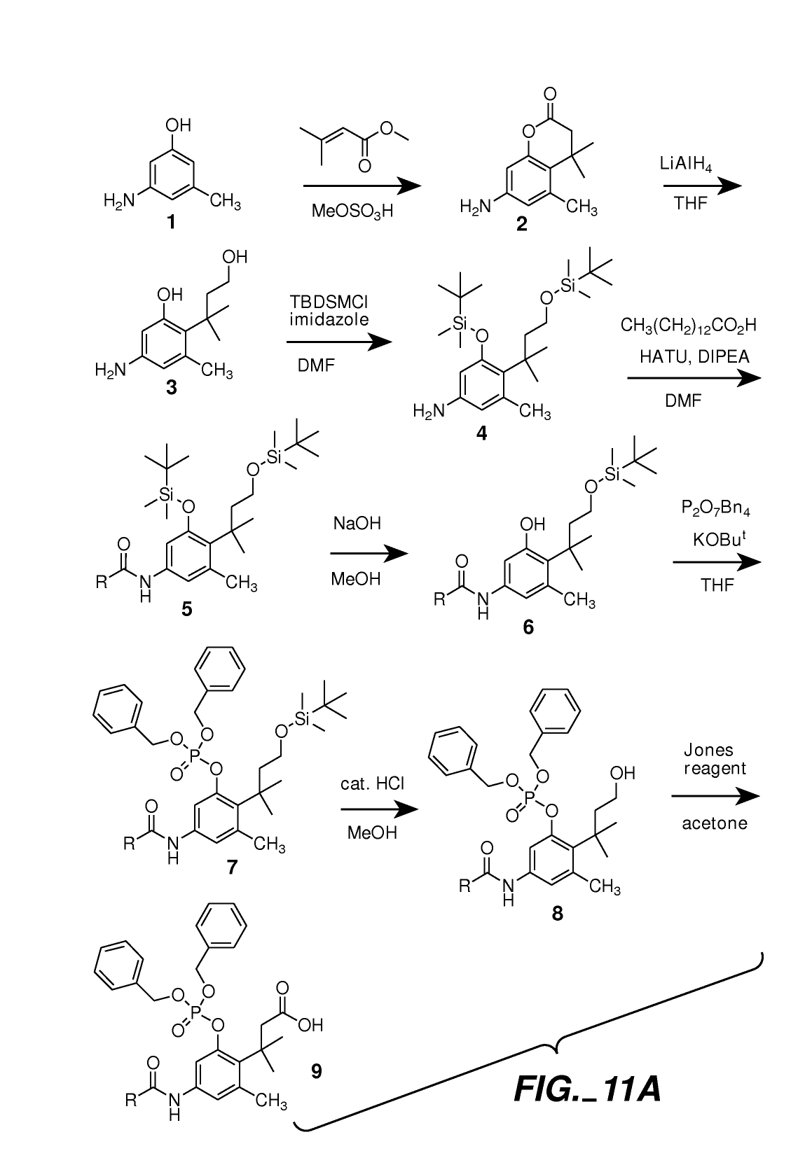

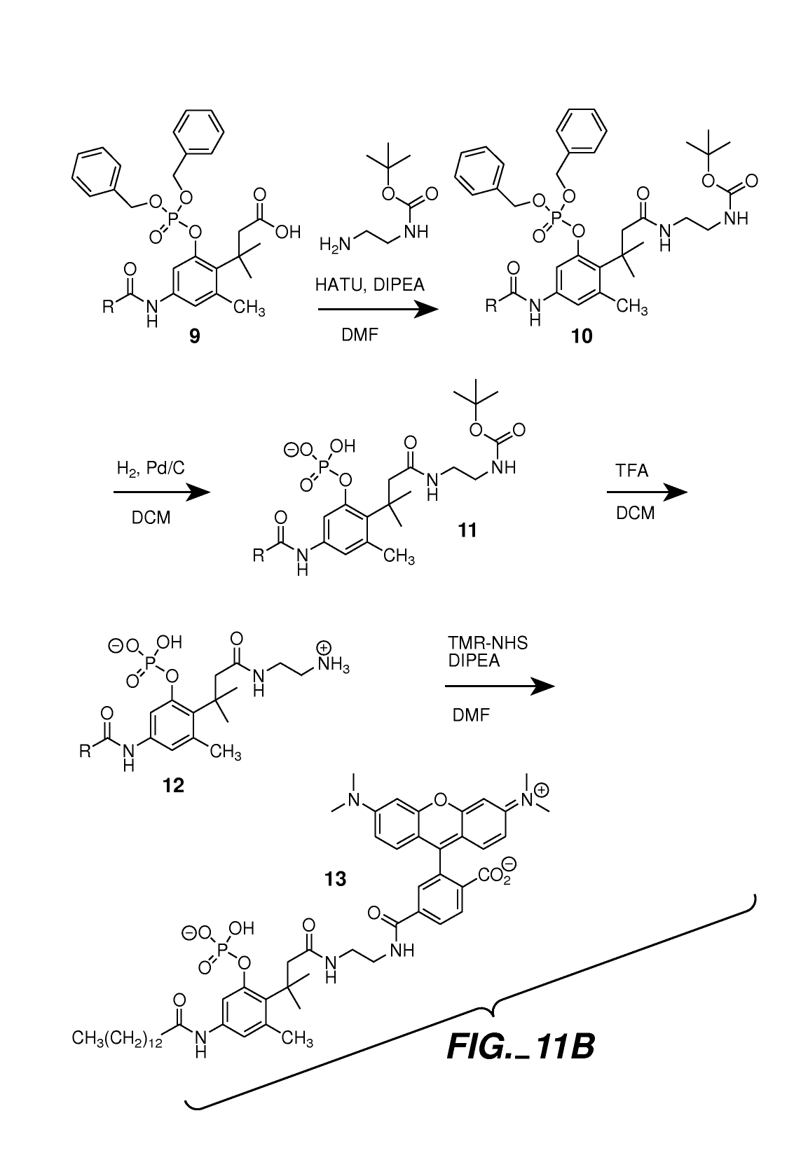

A synthetic scheme for the synthesis of a substrate compound capable of undergoing a ring closure elimination reaction, i.e. a trimethyl lock lactonization reaction, is illustrated in FIGS. 11A-11B. Referring to FIGS. 11A-11B, compound 1 can be reacted with methyl 3,3-dimethylacrylate in methanesulfonic acid to give compound 2. Reduction of 2 with lithium aluminum hydride can give the diol 3. The phenol and alkyl alcohol can be protected with tert-butyldimethylsilyl chloride and imidazole to give 4. The aniline group can be reacted with myristic acid under standard peptide coupling conditions to give amide 5. Selective hydrolysis of the phenolic silyl ether can be performed under basic conditions to give 6. Phosphorylation of 6 with tetrabenzyl pyrophosphate and potassium tert-butoxide can give 7. The alkyl silyl ether can be hydrolysed with catalytic acid in methanol to give 8. Oxidation of the alcohol with Jones reagent in acetone can give 9. Coupling of mono BOC protected ethylenediamine with 9 can be performed under standard peptide coupling conditions. Catalytic hydrogenation of 10 can cleave the benzyl protecting groups on the phosphate. Trifluoacetic acid treatment of 11 can cleave the BOC protecting group to give 12. Tetramethylrhodamine succinimidyl ester can be coupled with 12 under basic conditions to give the final product 13.

Skilled artisans will appreciate that any one of the hydrophobic moieties, fluorescent moieties and trigger moieties described herein can be used with the various substrate compounds illustrated in FIGS. 3A-4B. Additionally, the various illustrated features can be readily “mixed and matched” to provide other specific embodiments of exemplary substrate compounds.

### 6.6 Methods

The sample to be tested may be any suitable sample selected by the user. The sample may be naturally occurring or man-made. For example, the sample may be a blood sample, tissue sample, cell sample, buccal sample, skin sample, urine sample, water sample, or soil sample. The sample can be from a living organism, such as a eukaryote, prokaryote, mammal, human, yeast, or bacterium. The sample may be processed prior to contact with a substrate described herein by any method known in the art. For example, the sample may be subjected to a precipitation step, column chromatography step, heat step, etc.

The reaction mixture typically includes a buffer, such as a buffer described in the “Biological Buffers” section of the 2000-2001 Sigma Catalog. Exemplary buffers include MES, MOPS, HEPES, Tris (Trizma), bicine, TAPS, CAPS, and the like. The buffer is present in an amount sufficient to generate and maintain a desired pH. The pH of the reaction mixture is selected according to the pH dependency of the activity of the enzyme to be detected. For example, the pH can be from 2 to 12, from 4 to 11, or from 6 to 10. The reaction mixture also contains any necessary cofactors and/or cosubstrates for the enzyme. Additional mixture components are discussed in Section IV below. In one embodiment, the reaction mixture does not contain detergent or is substantially free from detergents.

In some embodiments, it may be desirable to keep the ionic strength as low as reasonably possible to help avoid masking charged groups in the reaction product, so that micelle formation of product molecules remains disfavored and destabilized. For example, high salt concentration (e.g., 1 M NaCl) may be inappropriate. In addition, it may be desirable to avoid high concentrations of certain other components in the reaction mixture that can also adversely affect the fluorescence properties of the product. Guidance regarding the effects of ionic species, such as metal ions, can be found in Surfactants and Interfacial Phenomena, 2nd Ed., M. J. Rosen, John Wiley & Sons, New York (1989), particularly chapter 3.

In some embodiments, methods are provided for screening for enzyme activity. In these embodiments, a sample that contains, or may contain, a particular enzyme activity is mixed with a substrate compound described herein, and the fluorescence is measured to determine whether an increase in fluorescence has occurred. Screening may be performed on numerous samples simultaneously in a multi-well or multi-reaction plate or device to increase the rate of throughput. [Kcat and Km may be determined by standard methods, as described, for example, in Fersht, Enzyme Structure and Mechanism, 2nd Edition, W.H. Freeman and Co., New York, (1985)).

In some embodiments, the reaction mixture may contain two or more different enzymes. This may be useful, for example, to screen multiple enzymes simultaneously to determine if at least one of the enzymes has a particular enzyme activity.

The substrate specificity of an enzyme can be determined by reacting an enzyme with different substrates having different enzyme recognition moieties, and the activity of the enzyme toward the substrates can be determined based on an increase in their fluorescence. For example, by reacting an enzyme with several different substrates having several different protease recognition moieties, a consensus sequence for preferred substrates of a protease can be prepared.

Each different substrate may be tested separately in different reaction mixtures, or two or more substrates may be present simultaneously in a reaction mixture. In embodiments in which the different substrates are present simultaneously in the reaction mixture, the substrates can contain the same fluorescent moiety, in which case the observed fluorescent signal is the sum of the signals from enzyme reaction with both substrates. Alternatively, the different substrates can contain different, fluorescently distinguishable fluorescent moieties that allow separate monitoring and/or detection of the reaction of enzyme with each different substrate simultaneously in the same mixture. The fluorescent moieties can be selected such that all or a subset of them are excitable by the same excitation source, or they may be excitable by different excitation sources. They can also be selected to have additional properties, such as, for example, the ability to quench one another when in close proximity thereto, by, for example, collisional quenching, FRET or another mechanism (or combination of mechanisms).

Although not necessary for operation of the methods, the assay mixture may optionally include one or more quenching compounds designed to quench the fluorescence of the fluorescent moiety of the substrate (and/or plurality of substrates when more than one substrate is present in the mixture). In some embodiments, such quenching molecules generally comprise a hydrophobic moiety capable of integrating the quenching compound into a micelle and a quenching moiety. The hydrophobic moiety can be any moiety capable of integrating the compound into a micelle, and as specific non-limiting exemplary embodiments, can comprise any of the hydrophobic moieties described previously in connection with the substrate compounds utilizing a linker moiety that fragments via an elimination reaction.

The quenching moiety can include any moiety capable of quenching the fluorescence of the fluorescent moiety of the enzyme substrate used in the assay (or one or more of the substrates if a plurality of substrates are used). Compounds capable of quenching the fluorescence of the various different types of fluorescent dyes discussed above, such as xanthene, fluorescein, rhodamine, cyanine, phthalocyanine and squaraine dyes, are well-known. Such quenching compounds can be non-fluorescent (also referred to as “dark quenchers” or “black hole quenchers”) or, alternatively, they may themselves be fluorescent. Examples of suitable non-fluorescent dark quenchers that can comprise the quenching moiety include, but are not limited to, Dabcyl, Dabsyl, the various non-fluorescent quenchers described in U.S. Pat. No. 6,080,868 (Lee et al.) and the various non-fluorescent quenchers described in WO 03/019145 (Ewing et al.). Examples of suitable fluorescent quenchers include, but are not limited to, the various fluorescent dyes described above in connection with the substrate compounds.

The ability of a quencher to quench the fluorescence of a particular fluorescent moiety may depend upon a variety of different factors, such as the mechanisms of action by which the quenching occurs. The mechanism of the quenching is not critical to success, and may occur, for example, by collision, by FRET, by another mechanisms or combination of mechanisms. The selection of a quencher for a particular application can be readily determined empirically. As a specific example, the dark quencher Dabcyl and the fluorescent quencher TAMRA have been shown to effectively quench the fluorescence of a variety of different fluorophores. In a specific embodiment, a quencher can be selected based upon its spectral overlap properties spectral overlap with the fluorescent moiety. For example, a quencher can be selected that has an absorbance spectrum that sufficiently overlaps the emission spectrum of the fluorescent moiety such that the quencher quenches the fluorescence of the fluorescent moiety are in close proximity to one another, such as when the quencher molecule and substrate compound are integrated into the same micelle.

In embodiments in which a plurality of substrates are present in the assay, such as the multiplexed embodiments described above, it may be desirable to select a quenching moiety that can quench the fluorescence of the fluorescent moieties of all of the substrates present in the assay.

The hydrophobic and quenching moieties can be connected in any way that permits them to perform their respective functions. In some embodiments, only one hydrophobic moiety is linked either directly or via a linker to a quenching moiety. In other embodiment, two hydrophobic moieties may be linked either directly or via a linker to a quenching moiety. As a specific example, one hydrophobic moiety may be linked directly to the quenching moiety without the aid of a linker. Non-limiting examples of such quenching compounds include molecules in which a dye (e.g. a rhodamine or fluorescein dye) which contains a primary amino group (or other suitable group) is acylated with a fatty acid. As another specific example, the linkage may be mediated by way of a linker moiety, such as described above. As a specific example, the quencher molecule can be a derivative or analog of any of the substrate compounds described herein in which the fluorescent moiety is replaced with a quenching moiety and the trigger moiety is modified such that it is not recognized by the enzyme(s) being assayed in the sample.

In addition, the methods can include means of detecting, screening for, and/or characterizing inhibitors, activators, and/or modulators of enzyme activity. For example, methods for detecting screening for, and/or characterizing inhibitors, activators, and/or modulators of enzyme activity can be performed by forming reaction mixtures containing such known or potential inhibitors, activators, and/or modulators and determining the extent of increase or decrease (if any) in fluorescence signal relative to the signal that is observed without the inhibitor, activator, or modulator. Different amounts of these substances can be tested to determine parameters such as Ki (inhibition constant), K_{H} (Hill coefficient), Kd (dissociation constant) and the like to characterize the concentration dependence of the effect that such substances have on enzyme activity.

Detection of fluorescent signal can be performed in any appropriate way. Advantageously, the substrate compounds described herein can be used in a continuous monitoring phase, in real time, to allow the user to rapidly determine whether enzyme activity is present in the sample, and optionally, the amount or specific activity of the enzyme. The fluorescent signal is measured from at least two different time points, usually until an initial velocity (rate) can be determined. The signal can be monitored continuously, periodically, or at several selected time points. Alternatively, the fluorescent signal can be measured in an end-point embodiment in which a signal is measured after a certain amount of time, and the signal is compared against a control signal (before start of the reaction), threshold signal, or standard curve.

### 6.7 Kits

Also provided are kits for making the substrate compound-containing micelles and/or for carrying out the various methods described herein. In one embodiment, the kit comprises a substrate compound comprising a hydrophobic moiety, a fluorescent moiety, a trigger moiety and a linker moiety. The kit may optionally comprise a quenching molecule and/or additional components for making the substrate compound-containing micelles. In one embodiment, the substrate compound comprising the hydrophobic moiety, fluorescent moiety, trigger moiety, linker moiety and optional quenching molecule and/or other components are packaged in a form such that they can be used to make substrate compound-containing micelles. In some embodiments, the substrate compound comprising the hydrophobic moiety, fluorescent moiety, trigger moiety, linker moiety and optional quenching molecule and other components are provided in a kit in the form of pre-formed lyophilized micelles that can be reconstituted for use, or in the form of pre-formed micelles in solution.

The kit may also comprise a binding assay buffer, or a component thereof. The buffer may be provided in a container in dry or liquid form. The choice of a particular buffer may depend on various factors, such as the pH optimum for the binding reaction, and the solubility and fluorescence properties of the fluorescent moiety of the amphiphilic molecule. In some embodiments, the buffer is provided as a stock solution having a pre-selected pH and buffer concentration. Upon mixture with the sample, the buffer produces a final pH that is suitable for the binding or modulator assays, as discussed above. In addition, the kit may comprise other components that are beneficial to the activity of the modification agent, such as salts (e.g., KCl, NaCl, or NaOAc, CaCl₂, MgCl₂, MnCl₂, ZnCl₂) and/or other components that may be useful for a particular assay. These other components can be provided separately from each other, such as in separate containers, or mixed together in dry or liquid form.

Unless defined otherwise, all technical and scientific terms used herein have the same meaning as commonly understood by one of ordinary skill in the art to which the compositions and methods described herein belong. Unless mentioned otherwise the techniques employed or contemplated herein are standard methodologies well known to one of ordinary skill in the art. The materials, methods and examples are illustrative only and not limiting.

All numerical ranges in this specification are intended to be inclusive of their upper and lower limits.

Other features of the methods and compositions described herein will become apparent in the course of the following descriptions of exemplary embodiments which are given for illustration, and are not intended to be limiting thereof.

### 7. EXAMPLE

### 7.1 Preparation of Compound 7, FIG. 5B

A prophetic example for the synthesis of compound 7 is illustrated in FIGS. 5A-5B. Referring to FIG. 5A, bromo 2,3,4,6-tetra-O-acetyl-α-D-galactopyranoside (4.0 g, 24 mmol, Toronto Research Chemicals catalogue #B687000) and 4-hydroxy-3-nitrobenzaldehyde (10 g, 24 mmol, Aldrich catalogue #14, 432-0) can be dissolved in acetonitrile (200 ml). Silver (I) oxide (25 g, 108 mmol) can be added and the suspension stirred at room temperature for 3 hours. The reaction mixture can be filtered with suction through a pad of celite, the filtrate collected and the solvent evaporated. The crude product can be purified by silica gel chromatography eluting with a 98:2 mixture of dichloromethane (DCM) and methanol (MeOH). A pale yellow foam (1, 10 g, 20 mmol, 83%) can be obtained after collecting the fractions and evaporating the solvent.

Compound 1 (3.4 g, 6.8 mmol) can be dissolved in DCM (150 ml). The solution can be sparged with argon for 10 min and then 10% Pd/C (0.5 g) can be added. The flask can be charged with hydrogen and shaken with a Parr apparatus. After 3 hr the reaction mixture can be filtered with suction through a pad of celite The filtrate can be concentrated and the crude product can be purified by silica gel chromatography eluting with a 98:2 mixture of DCM and MeOH. A colorless foam (2, 2.5 g, 5.3 mmol, 78%) can be obtained after collecting the fractions and evaporating the solvent.

Compound 2 (2.9 g, 6.2 mmol) can be dissolved in dry dimethylformamide (DMF, 20 ml). Imidazole (0.63 g, 9.3 mmol) and tert-butyldimethylsilyl chloride (1.4 g, 9.3 mmol) can be added. After 30 min most of the solvent can be evaporated and water (50 ml) followed by ether (50 ml) can be added. The layers can be separated and the ether layer can be washed with water (25 ml) followed by brine (25 ml). The solvent can be evaporated and the crude product can be purified by silica gel chromatography eluting with a 100:1 mixture of DCM and MeOH. A colorless oil (3, 4.5 g, 7.7 mmol, 67%) can be obtained after collecting the fractions and evaporating the solvent.

Compound 3 (4.5 g, 7.7 mmol) and myristic acid (1.8 g, 7.7 mmol) can be dissolved in DMF (20 ml). N,N-diisopropylethylamine (DIPEA, 0.99 g, 7.7 mmol) can be added followed by N—[(dimethylamino)-1H-1,2,3-triazolo[4,5-b]pyridin-1-ylmethylene]-N-methylmethanaminium hexafluorophosphate N-oxide (HATU, 2.9 g, 7.7 mmol). After 30 min most of the solvent can be evaporated and water (50 ml) followed by ether (50 ml) can be added. The layers can be separated and the ether layer can be washed with water (25 ml) followed by brine (25 ml). The solvent can be evaporated and the crude product can be purified by silica gel chromatography eluting with a 100:1 mixture of DCM and MeOH. A colorless solid (4, 4.8 g, 6 mmol, 78%) can be obtained after collecting the fractions and evaporating the solvent.

Compound 4 (2.4 g, 3 mmol) can be dissolved in a solution of HC in MeOH (60 mM, 16.7 ml, 1 mmol HCl). After 30 min the acid can be neutralized with NaHCO₃ (84 mg, 1 mmol) in water (3 ml). Most of the solvent can be evaporated and water (50 ml) followed by ether (50 ml) can be added. The layers can be separated and the ether layer can be washed with water (25 ml) followed by brine (25 ml). The solvent can be evaporated and the crude product can be purified by silica gel chromatography eluting with a 100:1 mixture of DCM and MeOH. A colorless solid (compound 5, 1.6 g, 2.4 mmol, 79%) can be obtained after collecting the fractions and evaporating the solvent.

Compound 5 (16 mg, 23 μmol) can be dissolved in warm acetonitrile (2 ml). N,N′-disuccinimidyl carbonate (DSC, 6 mg, 23 μmol) and DIPEA (6 mg, 8 μl, 46 μmol) can then be added. After 1 h 5-(aminomethyl)fluorescein hydrochloride (9 mg, 23 mol) can be added. The crude product 6 can be used in the next step.

Ammonium hydroxide solution (15 M, 1 ml) can be added to the above crude product 6 and left to sit overnight. The reaction mixture can be diluted with water (18 ml) and purified by reverse phase HPLC eluting with a 2:3 mixture of triethylammonium acetate buffer (100 mM) and methanol. Fractions can be combined and most of the solvent evaporated. The product can be desalted on a short plug of C18 reverse phase media. The product should be obtained as an orange solid (7, 5 mg, 5 μmol, 21%).

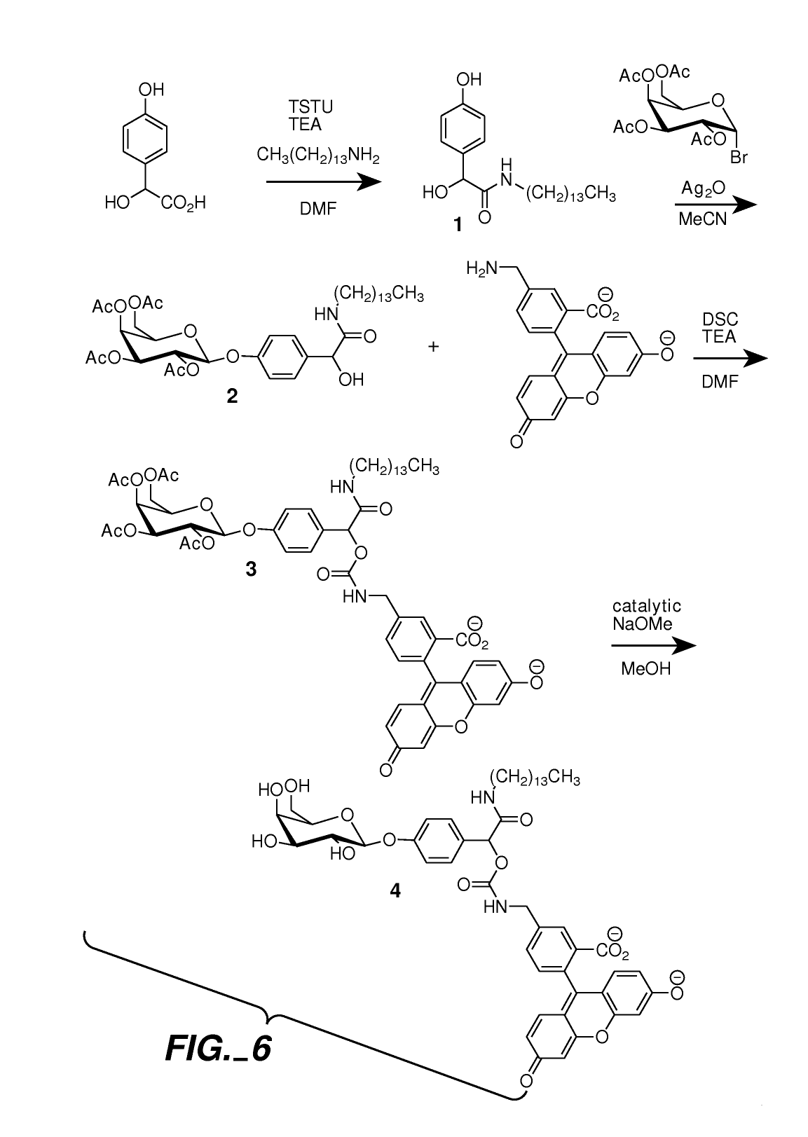

### 7.2 Preparation of Compound 4, FIG. 6

Referring to FIG. 6, 4-Hydroxymandelic acid (Aldrich catalogue #16, 832-7) can be coupled with 1-tetradecylamine under standard peptide coupling conditions to yield amide 1. The phenolic hydroxyl group can be selectively glycosylated under Koenig-Knorr conditions to give β-glycoside 2. The benzylic hydroxyl group of compound 2 can be reacted with N,N′-disuccinimidyl carbonate (DSC) or other phosgene synthetic equivalent to give the mixed carbonate. 5-Aminomethyl fluorescein (Molecular Probes catalogue #A-1353) can be coupled with the mixed carbonate under basic conditions to give carbamate 3. The four acetate protecting groups on the sugar can be hydrolysed with catalytic sodium methoxide in methanol to give compound 4.

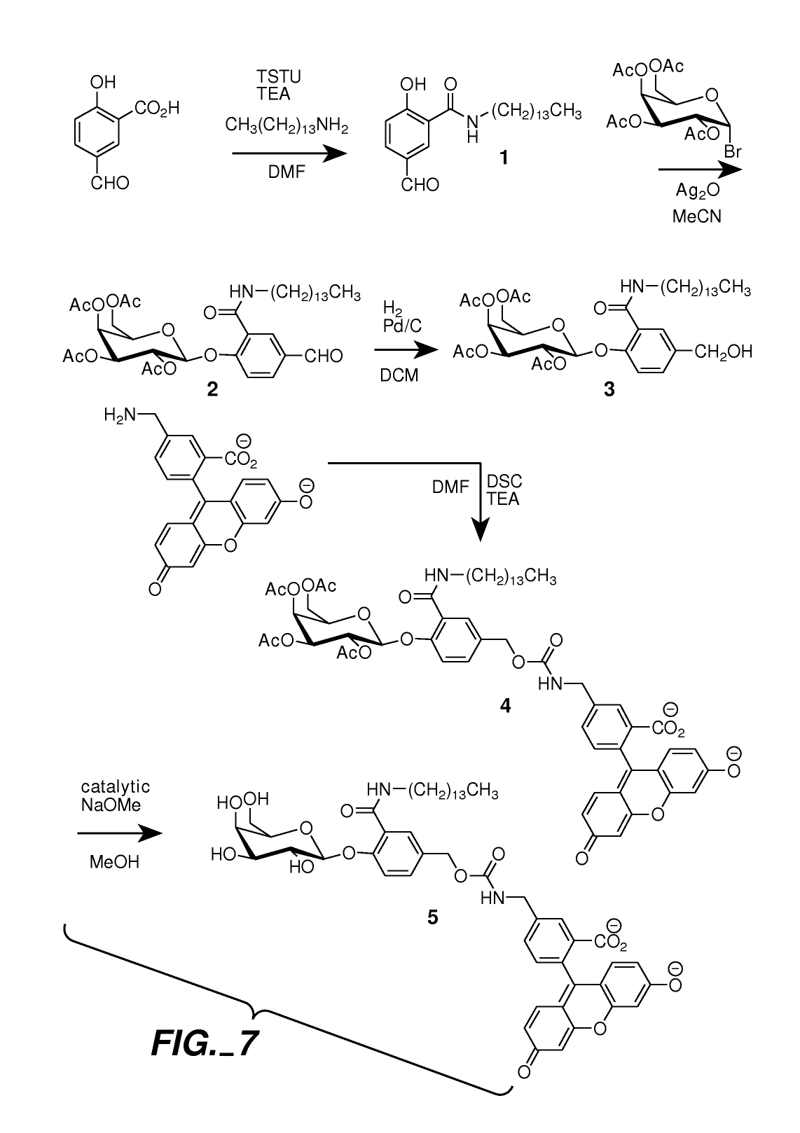

### 7.3 Preparation of Compound 5, FIG. 7

Referring to FIG. 7, 5-Formylsalicylic acid (Aldrich catalogue #F1,760-1) can be coupled with 1-tetradecylamine under peptide coupling conditions to give amide 1. The phenolic hydroxyl group can be glycosylated under Koenig-Knorr conditions to give β-glycoside 2. The benzaldehyde group can be reduced under catalytic hydrogenation conditions to give compound 3. The benzylic hydroxyl group of compound 3 can be reacted with N,N′-disuccinimidyl carbonate (DSC) or other phosgene synthetic equivalent to give the mixed carbonate. 5-Aminomethyl fluorescein (Molecular Probes catalogue #A-1353) can be coupled with the mixed carbonate under basic conditions to give carbamate 4. The four acetate protecting groups on the sugar can be hydrolysed with catalytic sodium methoxide in methanol to give compound 5.

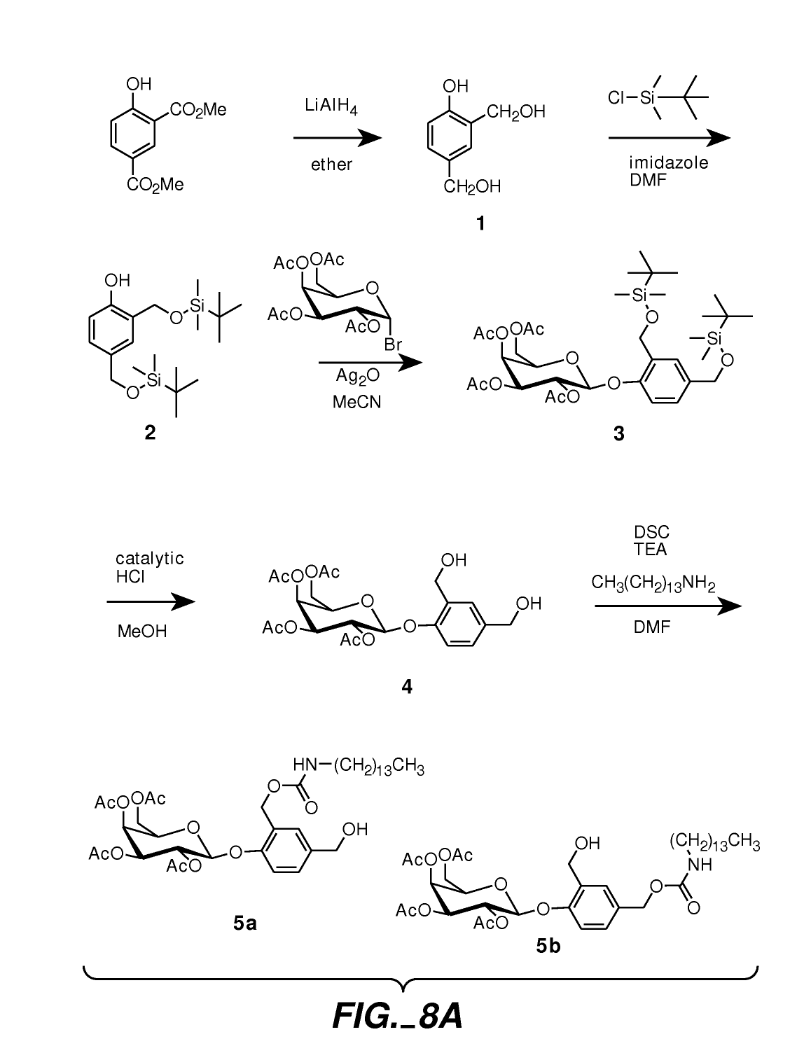

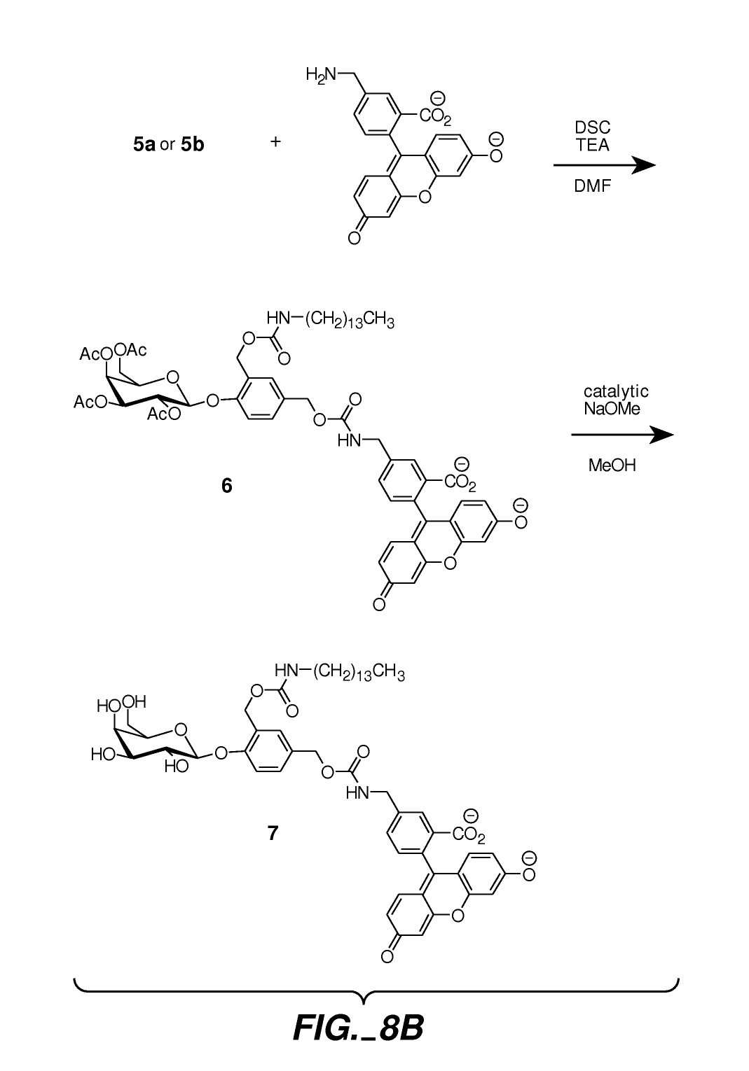

### 7.4 Preparation of Compound 7, FIG. 8

Referring to FIG. 8A, dimethyl 4-hydroxyisophthalate (Aldrich catalogue #541095) can be reduced with lithium aluminum hydride to give the triol 1. The benzylic alcohols can be selectively protected with tert-butyldimethylsilyl chloride to give compound 2. The phenol can be glycosylated under Koenig-Knorr conditions to give β-glycoside 3. The silyl protecting groups can be hydrolysed with catalytic hydrochloric acid in methanol to give diol 4. One equivalent of N,N′-disuccinimidyl carbonate (DSC) or other phosgene synthetic equivalent can be added to compound 4 to give a mixture of two regioisomeric monocarbonates. 1-Tetradecylamine can be added to the mixture of monocarbonates to give a mixture of regioisomeric monocarbamates 5a,b. The regioisomers may be separated by chromatography if desired. One equivalent of N,N′-disuccinimidyl carbonate (DSC) or other phosgene synthetic equivalent can be added to compound 5 to give a mixed carbonate. 5-Aminomethyl fluorescein (Molecular Probes catalogue #A-1353) can be coupled with the mixed carbonate under basic conditions to give carbamate 6. The four acetate protecting groups on the sugar can be hydrolysed with catalytic sodium methoxide in methanol to give compound 7.

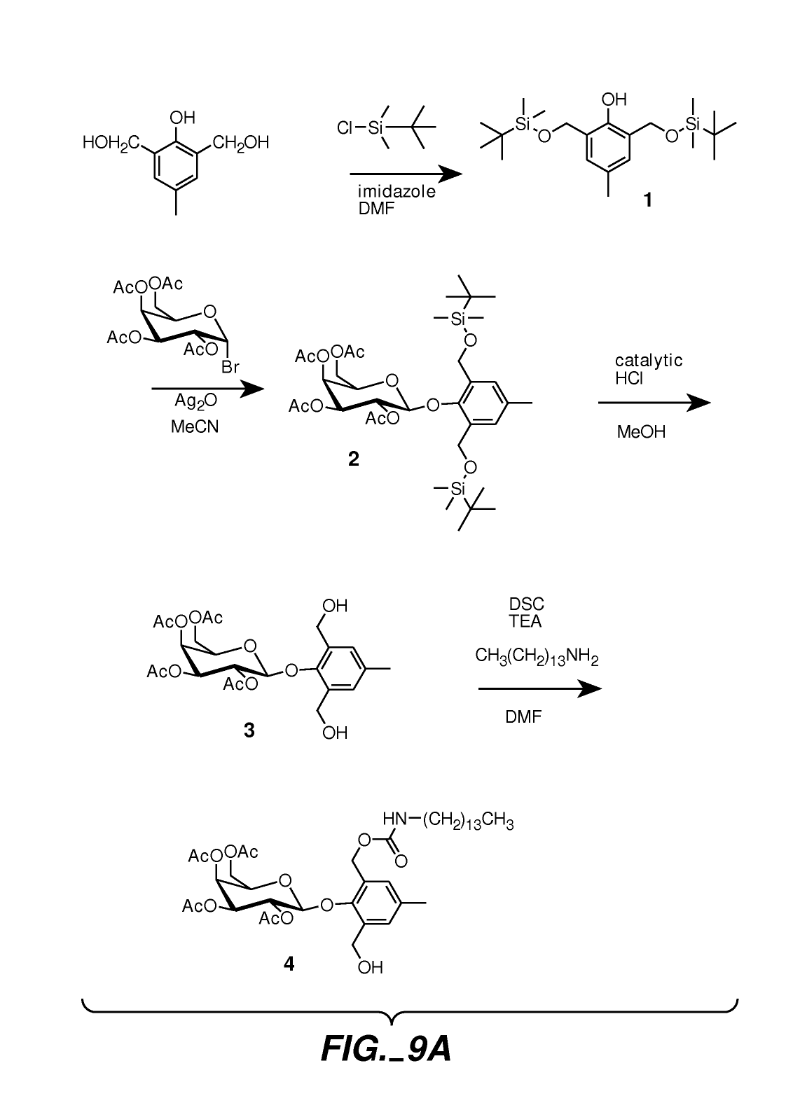

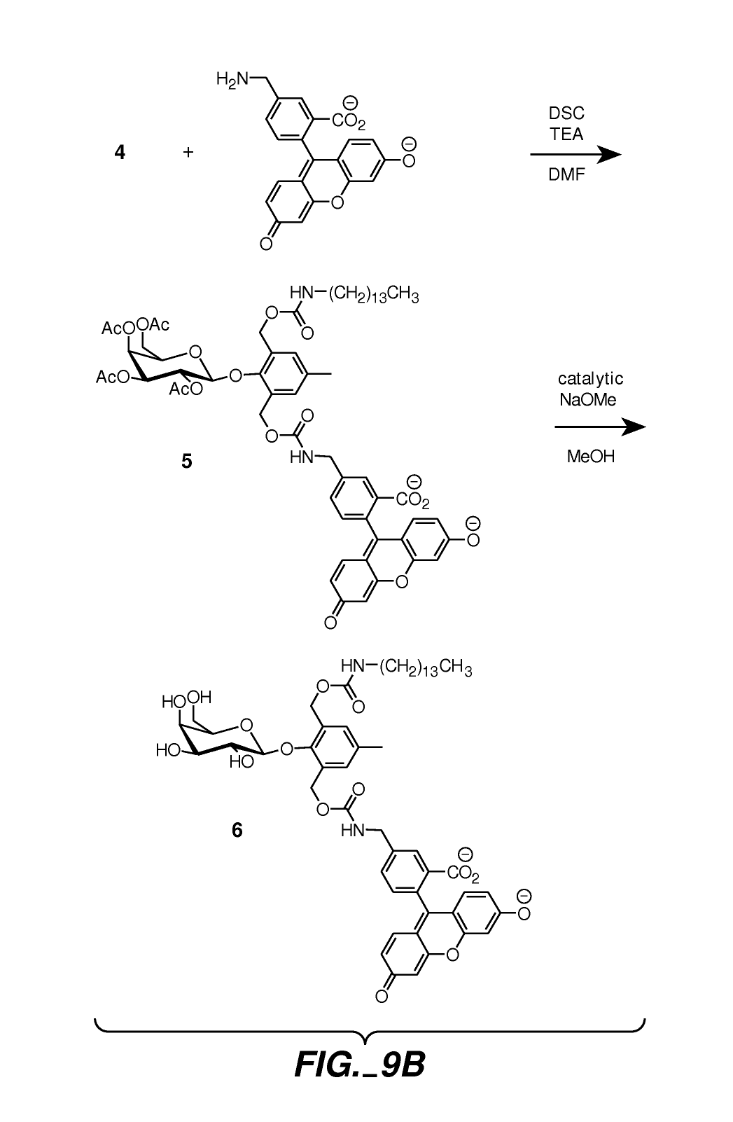

### 7.5 Preparation of Compound 6, FIG. 9B

Referring to FIG. 9A, 2,6-Bis(hydroxymethyl)-p-cresol (Aldrich catalogue #22, 752-8) can be selectively protected with two equivalents of tert-butyldimethylsilyl chloride to give 1. The phenol can be glycosylated under Koenig-Knorr conditions to give β-glycoside 2. The silyl protecting groups can be hydrolysed with catalytic hydrochloric acid in methanol to give diol 3. One equivalent of N,N′-disuccinimidyl carbonate (DSC) or other phosgene synthetic equivalent can be added to compound 3 to give a mixed carbonate. 1-Tetradecylamine can be added to the mixed carbonate under basic conditions to give carbamate 4. One equivalent of N,N′-disuccinimidyl carbonate (DSC) or other phosgene synthetic equivalent can be added to compound 4 to give a mixed carbonate. 5-Aminomethyl fluorescein (Molecular Probes catalogue #A-1353) can be coupled with the mixed carbonate under basic conditions to give carbamate 5. The four acetate protecting groups on the sugar can be hydrolysed with catalytic sodium methoxide in methanol to give compound 6.

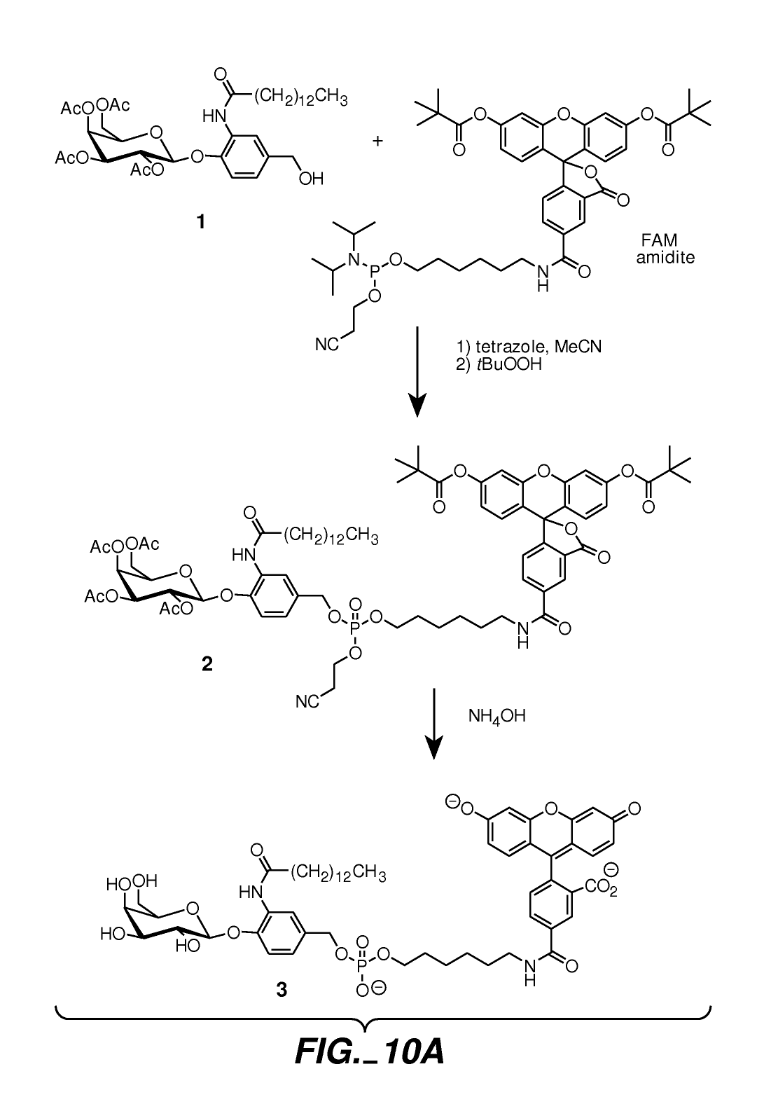

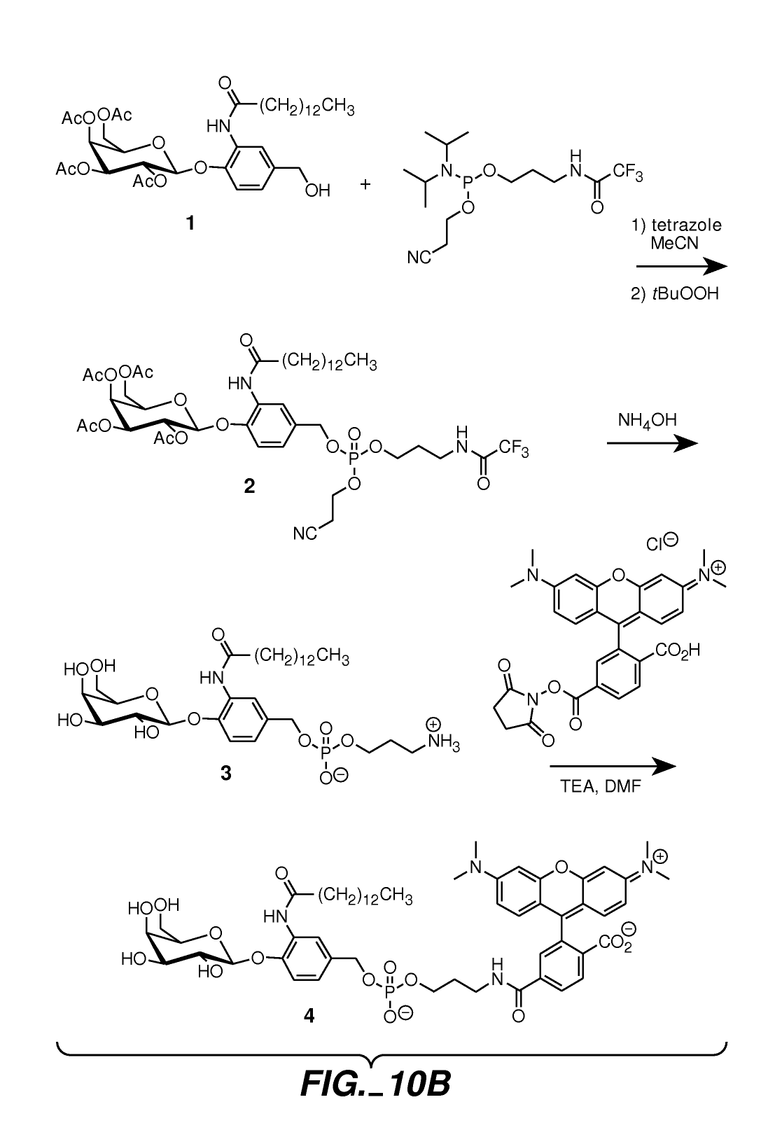

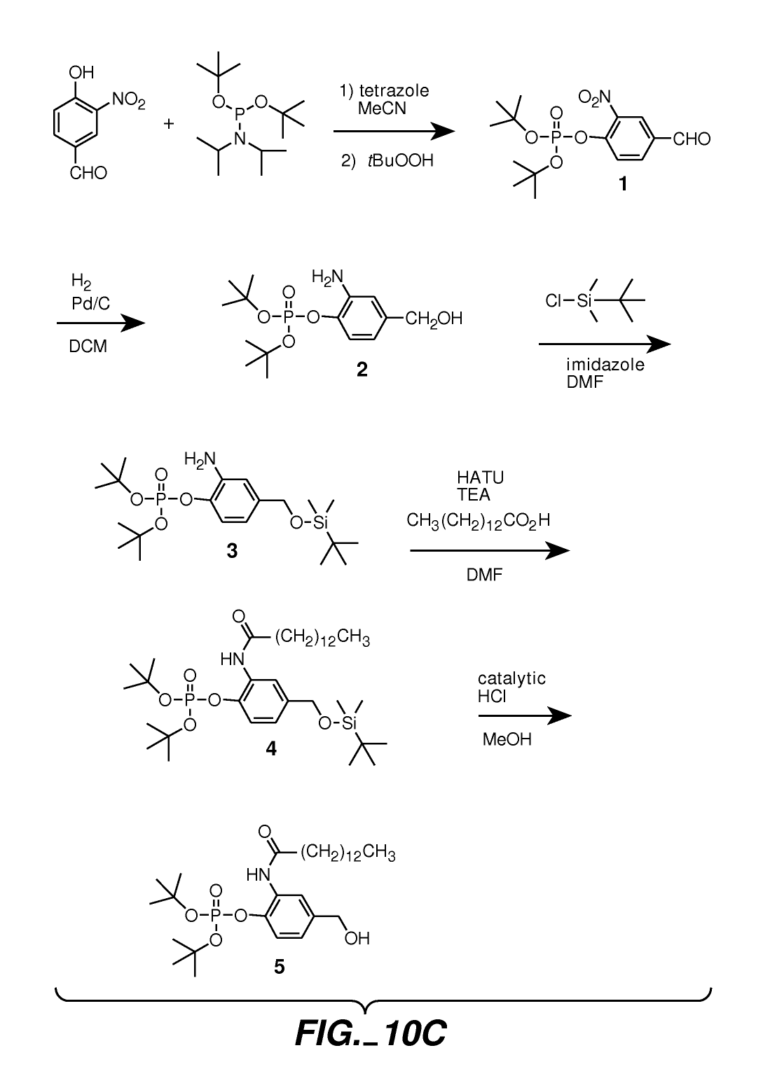

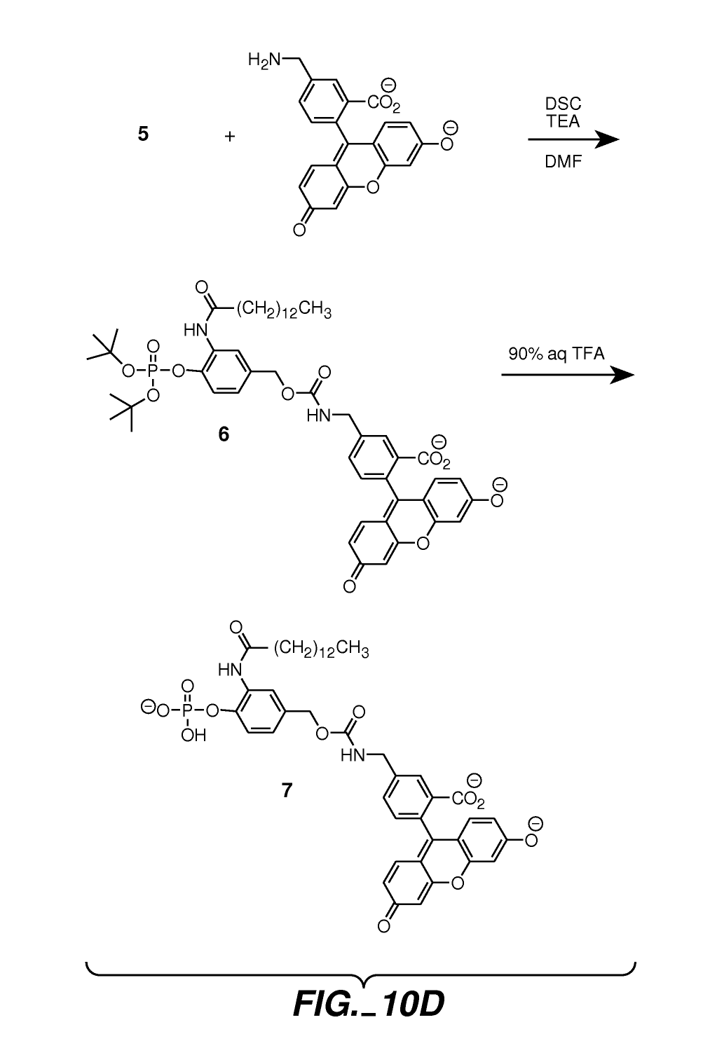

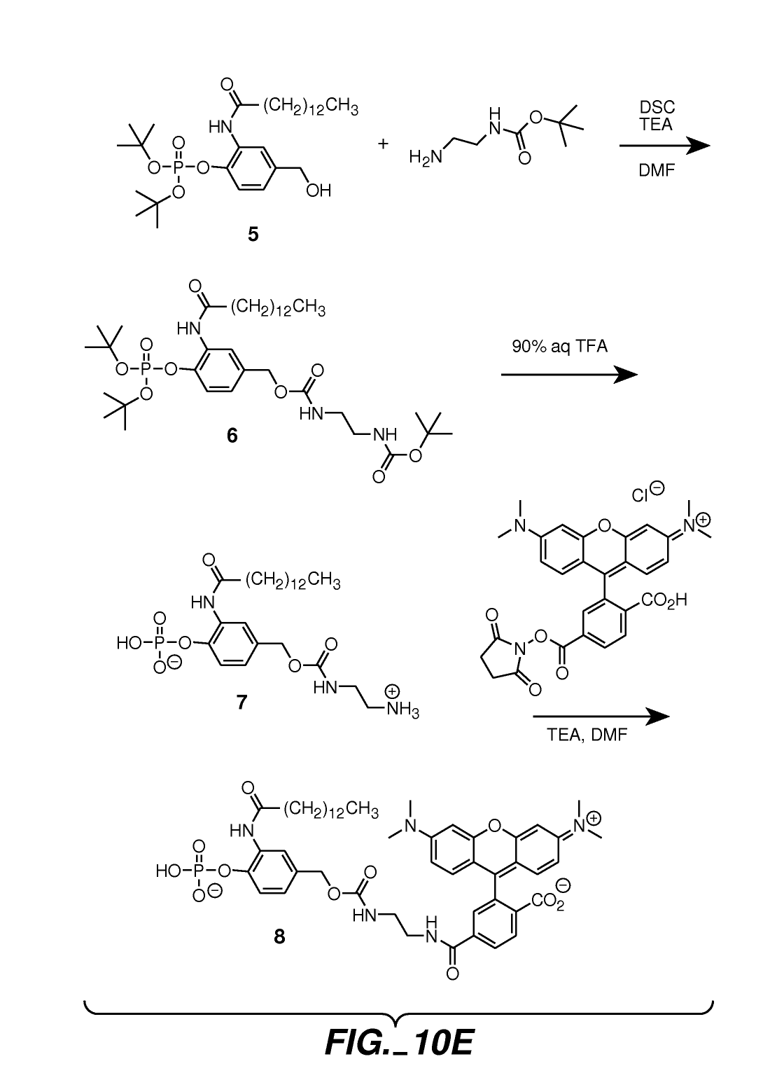

### 7.6 Preparation of Compound 3, FIG. 10A

Referring to FIG. 10A, the benzylic alcohol of compound 1 can be reacted with FAM® phosphoramidite (Applied Biosystems catalogue #401527) under standard tetrazole coupling conditions. The phosphite can be oxidized with tert-butylhydroperoxide to give the phosphate 2. Concentrated ammonium hydroxide can be used to cleave the cyanoethyl, four acetyl, and two pivaloyl protecting groups to give compound 3.

### 7.7 Preparation of Compound 4, FIG. 10B

Referring to FIG. 10B, Compound 1 can be reacted with TFA aminolink phosphoramidite (Applied Biosystems catalogue #402872) under standard tetrazole conditions. The phosphite can be oxidized with tert-butylhydroperoxide to give phosphate 2. Concentrated ammonium hydroxide can be used to cleave the trifluoroacetyl, cyanoethyl, and four acetyl protecting groups to give 3. Carboxytetramethylrhodamine succinimidyl ester (Molecular Probes catalogue #C2211) can be coupled to the primary amine under basic conditions to give 4.

### 7.8 Preparation of Compound 7, FIG. 10D

Referring to FIG. 10C, 4-Hydroxy-3-nitrobenzaldehyde (Aldrich catalogue #14,432-0) can be reacted with di-tert-butyl-N,N-diisopropylphosphoramidite (Novabiochem catalogue #01-60-0031) to give a phosphite that can be subsequently oxidized to the phosphate with tert-butylhydroperoxide. The benzaldehyde and nitro groups of compound 1 can be reduced under catalytic hydrogenation conditions to give the aminoalcohol 2. The hydroxyl group can be protected as its tert-butyldimethylsilyl ether. Myristic acid can be coupled with the aniline under standard peptide coupling conditions to give 4. The silyl ether protecting group can be hydrolyzed with catalytic hydrochloric acid in methanol to give 5. The benzyl alcohol can be reacted with DSC or other phosgene synthetic equivalent to give the mixed carbonate. 5-Aminomethyl fluorescein (Molecular Probes catalogue #A-1353) can be added under basic conditions to give the carbamate 6. The two tert-butyl protecting groups on the phosphate can be hydrolysed with 90% aqueous trifluoroacetic acid to give 7.

### 7.9 Preparation of Compound 8, FIG. 10E

Referring to FIG. 10E, the benzyl alcohol of compound 5 can be reacted with DSC or other phosgene synthetic equivalent to give the mixed carbonate. N-Boc-ethylenediamine (Fluka catalogue #15369) can be added under basic conditions to give the carbamate 6. The two tert-butyl and boc protecting groups can be hydrolysed with 90% aqueous trifluoroacetic acid to give 7. Carboxytetramethylrhodamine succinimidyl ester (Molecular Probes catalogue #C2211) can be coupled to the primary amine under basic conditions to give 8.

1. A substrate compound comprising:
a) a hydrophobic moiety capable of integrating the substrate compound into a micelle;
b) a fluorescent moiety;
c) a trigger moiety; and
d) a linker linking the hydrophobic, fluorescent and trigger moieties that is capable of fragmenting to release the fluorescent moiety or the hydrophobic moiety when the trigger moiety is acted upon by a trigger agent selected from structures 4, 5, 6, and 7:

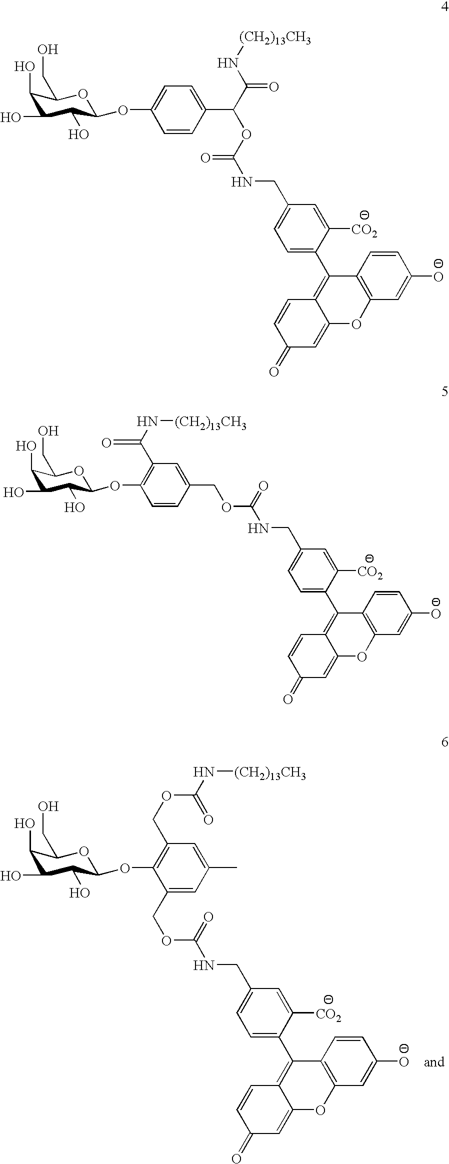

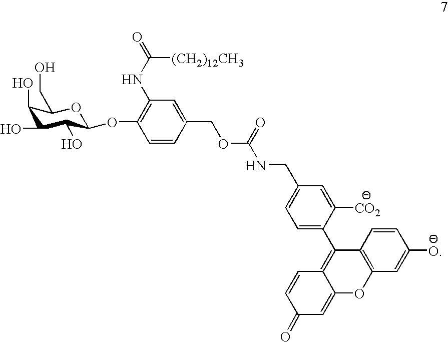

2. The substrate compound of claim 1 in which the trigger moiety comprises a substrate for a cleaving enzyme.

3. The substrate compound of claim 2 in which the cleaving enzyme is selected from a lipase, an esterase, a phosphatase, a protease, a glycosidase, a carboxypeptidase and a catalytic antibody.

4. The substrate compound of claim 2 in which the linker fragments via an elimination reaction selected from the group consisting of 1,4-, 1,6-, and 1,8-elimination reactions when the substrate is cleaved from the substrate compound by the cleaving enzyme.

5. The substrate compound of claim 4 in which the elimination is 1,4-elimination.

6-10. (canceled)

11. The substrate compound of claim 1 in which the linker fragments via elimination when the trigger moiety is acted upon by the trigger agent.

12-13. (canceled)

14. The substrate compound of claim 1 in which the fluorescent moiety comprises a fluorescent dye selected from a xanthene dye, a fluorescein dye, a rhodamine, a cyanine dye, a phthalocyanine dye, a squaraine dye and a bodipy dye.

15. The substrate compound of claim 1 in which the hydrophobic moiety comprises a hydrocarbon group containing from 6 to 30 carbon atoms.

16-39. (canceled)

40. A micelle comprising a plurality of substrate compounds of claim 1, each of which comprises:
a) a hydrophobic moiety capable of integrating the substrate compound into the micelle;
b) a fluorescent moiety;
c) a trigger moiety; and
d) a linker linking the hydrophobic, fluorescent and trigger moieties that is capable of fragmenting to release the fluorescent moiety or the hydrophobic moiety when the trigger moiety is acted upon by a trigger, wherein the fluorescence signals of the fluorescent moieties in the micelle are quenched as compared to their fluorescence signals when released from their respective substrate compounds.

41. The micelle of claim 40 in which each substrate compound of the plurality is the same.

42. The micelle of claim 40 which further comprises a plurality of quenching compounds, each of which comprises a hydrophobic moiety capable of integrating the quenching compound into the micelle and a quenching moiety capable of quenching the fluorescence signal of a fluorescent moiety in the micelle.

43. A micelle comprising:
a) a first substrate compound of claim 1 comprising a first hydrophobic moiety capable of integrating the first substrate compound into the micelle, a first fluorescent moiety, a first trigger moiety and a first linker linking the first hydrophobic, fluorescent and trigger moieties that is capable of fragmenting to release the first fluorescent moiety or first hydrophobic moiety from the micelle when the first trigger moiety is acted upon by a first trigger; and
b) a second substrate compound of claim 1 comprising a second hydrophobic moiety capable of integrating the second substrate compound into the micelle, a second fluorescent moiety, a second trigger moiety and a second linker linking the second hydrophobic, fluorescent and trigger moieties that is capable of fragmenting to release the second fluorescent moiety or the second hydrophobic moiety from the micelle when the second trigger moiety is acted upon by a second trigger,
wherein the first and second trigger moieties are triggered by different triggers, the fluorescence signals of the first and second fluorescent moieties are resolvable from one another and the fluorescence signals of the first and second fluorescent moieties in the micelle are quenched as compared to their fluorescent signals when released from the micelle.

44. The substrate compound of claim 3 in which the glycosidase is selected from beta-galactosidase and beta-glucoronidase.

45. The substrate compound of claim 44 in which the glycosidase is beta-galatosidase.

46. The substrate compound of claim 44 in which the glycosidase is beta-glucoronidase.
